(19) United States
(12) Patent Application Publication
(10) Pub. No.: US 2008/0159994 A1
(43) Pub. Date: Jul. 3, 2008

(54) METHODS FOR EMBRYONIC STEM CELL CULTURE

(76) Inventors: Sakis Mantalaris, London (GB); Wesley Randle, Babraham (GB)

Correspondence Address: FROMMER LAWRENCE & HAUG, 745 FIFTH AVENUE- 10TH FL., NEW YORK, NY, 10151, US

(21) Appl. No.: 11/830,378

(22) Filed: Jul. 30, 2007

Related U.S. Application Data
(63) Continuation-in-part of application No. PCT/GB2006/050026, filed on Jan. 30, 2006.
(60) Provisional application No. 60/647,461, filed on Jan. 28, 2005.

(30) Foreign Application Priority Data
Jan. 28, 2005 (GB) 0501637.3

(51) Int. Cl. A61K 35/12; C12N 5/08; C12N 5/06; A61P 19/10; A61P 35/00; C12M 1/00; A01N 1/00; A61P 19/00; C12Q 1/02; C12Q 1/68

(52) U.S. Cl. 424/93.7; 435/366; 435/377; 435/363; 435/325; 435/350; 435/352; 435/29; 435/6; 435/1.1; 435/289.1

(57) ABSTRACT

The invention relates to a method of cell culture comprising providing a pluripotent ES cell encapsulated within a support matrix to form a support matrix structure, maintaining the encapsulated cell in 3-D culture in maintenance medium, and optionally differentiating the encapsulated cell in 3-D culture in differentiation medium. The invention further relates to screening methods incorporating the use of encapsulated cells.

This application is a continuation-in-part application of international patent application Serial No. PCT/GB2006/050026 filed Jan. 30, 2006, which claims priority to U.S. provisional patent application Ser. No. 60/647,461 filed on Jan. 28, 2005, and to UK patent application Serial No. 0501637.3 filed on Jan. 28, 2005.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The invention relates to methods of culturing pluripotent cells to promote controlled self-renewal of the cells. The invention further provides integrated methods for expanding and differentiating homogeneous populations of cells from pluripotent cells. Additionally, the invention provides screening methods to identify conditions, media and stimuli that influence growth and differentiation of pluripotent cells, such as embryonic stem cells.

### BACKGROUND OF THE INVENTION

The term “stem cells” describes cells that can give rise to cells of multiple tissue types. There are different types of stems cells. A single totipotent cell is formed when a sperm fertilizes an egg, and has thereby has the capacity to form an entire organism. In the first hours after fertilization, this cell divides into identical totipotent cells. Approximately four days after fertilization and after several cycles of cell division, these totipotent stem cells begin to specialize. When totipotent cells become more specialized, they are then termed “pluripotent.” Pluripotent cells can be differentiated to every cell type in the body, but do not give rise to the placenta, or supporting tissues necessary for fetal development. Because the potential for differentiation of pluripotent cells is not “total,” such cells are not termed “totipotent” and they are not embryos. Pluripotent stem cells undergo further specialization into multipotent stem cells, which are committed to differentiate to cells of a particular lineage specialized for a particular function. Multipotent cells can be differentiated to the cell types found in the tissue from which they were derived; for example, blood stem cells can be differentiated only into red blood cells, white blood cells and platelets.

Pluripotent stem cells, which can include embryonic stem (ES) cells, embryonic germ (EG) cells, and multipotent stem cells, such as umbilical cord stem cells and adult stem cells, are powerful tools proposed for use in tissue engineering due to their ability to self-renew and their capacity for plasticity. Pluripotent stem cells such as ES cells can be induced to differentiate in vitro into multipotent cells of mesoderm, ectoderm and endoderm cell lineages. Mesodermal lineage cells, such as osteoblasts, chondrocytes and cardiomyocytes, are generated under the influence of osteogenic, chondrogenic, and myogenic supplements, respectively. At present, the use of pluripotent stem cells and multipotent cells in medicine is restricted by insufficient knowledge on formation of tissue-like structures and by the tendency to spontaneously differentiate towards different cell lineages; indeed this multi-lineage potential may represent a risk of heterotropic tissue formation. For clinical use, homogeneous cell populations with high purity may be necessary.

For clinical therapies using pluripotent cells to be effective, one prerequisite is the supply of an adequate number of cells for the relevant clinical application. Undifferentiated embryonic stem cells are a promising source for generation of key differentiated cell types, but for many undifferentiated cell populations, current culture methods are either not suitable for expansion or do not provide a useful yield of differentiated cells.

Current methods for maintenance of human ES (hES) cells require the use of feeder layers, feeder-conditioned media, or provision of human or animal cell extracts in the media to permit expansion of the hES cells and prevent spontaneous differentiation. Such methods are not suitable when it is subsequently proposed to use the cells in human therapy. The clinical application of hES cells requires methods of culturing the cells in standardized, well regulated environments in the absence of animal products (so called ‘xeno-free’ culture environments to eliminate the risk of disease transfer). In addition, methods of culturing hES cells in the absence of feeder or support cells are needed to eliminate the risk of contaminating the hES cell therapeutic product with the feeder cells or contaminants derived therefrom. Ideally, methods of producing sufficient numbers of hES cells should be standardized and regulatable. Such methods have not hitherto been available and the isolation and maintenance of hES cells using traditional methods is a highly skilled process not amenable to clinical application (1). Thus, there is a need to develop improved culture methods for expansion and, if desired, subsequent differentiation of hES cells.

Methods are known that can achieve the transition from undifferentiated murine embryonic stem cells (mES) to more differentiated cell types. However, in applying existing 2-D plate or flask culture protocols, the process is fragmented, involves high maintenance, is disruptive to the sample, and can have highly variable results.

Traditionally, embryonic stem culture protocols in 2-D cultures involve three distinct stages: (1) ES maintenance (i.e. self-renewal, also termed expansion, to form stem cell colonies), (2) initial differentiation leading to embryoid body (EB) formation, and (3) further lineage-specific differentiation. Each stage requires skilled manipulation and stage-specific protocols.

For ES maintenance, the original protocol involved ES cells that were isolated and co-cultured on feeder layers. It was subsequently found that conditioned media can be used instead of feeder layers (2; 3), and that for mES cells, LIF (a trophic factor secreted from feeders) could maintain pluripotency when supplied in purified form (4).

Assessment of ES cell pluripotency is performed by monitoring expression of the Octamer binding factor 3/4 (known as Oct-4). Oct-4 is a Pit-Oct-Unc (POU) family transcriptional regulator restricted to early embryos, germ-line cells, and undifferentiated EC (embryonic carcinoma), EG, and ES cells (5). Oct-4 expression in vivo is required for the development of pluripotent capacity of inner cell mass (ICM) cells (6) and, in vitro, it is chemostatically controlled for the maintenance of pluripotency (7).

In traditional differentiation methods, inner cell mass (ICM) derived embryonic stem cells are differentiated into various cell types via a stage in which an embryoid body (EB) is formed. Embryoid body formation, i.e. initial differentiation of ES cells, can be initiated by various stimuli, such as removal of feeder cells, removal of exposure to LIF (for murine ES cells), or removal of exposure to feeder-conditioned media. The EB suspension method developed for embryonal carcinoma (EC) cells (8) leads to formation of multi-differentiated structures, similar to post-implantation embryonic tissue, by formation of all three germ layers: mesoderm, ectoderm and endoderm (9). Within two to four days in suspension culture, ectoderm forms on the surface of the ICM, giving rise to structures termed “simple EBs.” At around day four of differentiation, a columnar epithelium with a basal lamina develops and a central cavity forms. These structures are termed “cystic EBs” and upon continued in vitro culture, endodermal and mesodermal cells appear (10).

Ectodermal cells are multipotent and can be differentiated into neural tissue, epithelium and dental tissue. Endodermal cells are multipotent and can be differentiated into the gastrointestinal tract, the respiratory tract and the endocrine glands. Mesodermal cells are multipotent and can be differentiated to haemopoietic and skeletal lineages, the latter including cardiomyogenic, chondrogenic and osteogenic cells. In the mesoderm, cardiogenic differentiation is known to be the first and predominant differentiation process. It is thought that cardiogenic differentiation may deter and retard other differentiation processes, such as chondrogenic and osteogenic differentiation.

Osteogenic differentiation, the in vitro formation of mineralized nodules that exhibit the morphological, ultrastructural and biochemical characteristics of woven bone formed in vivo, has been achieved by differentiation of functional osteoblasts in 2-D culture. However, 2-D culture performed in flasks and well-plates permits only a small number of cells to differentiate to the extent of being capable of organizing their extracellular matrix into a structure that resembles that of bone (11-13). Furthermore, 2-D culture is fragmented, labor intensive, and requires the “judgment” of the operator during the various culture steps involved.

Chondrogenic differentiation, the in vitro formation of cartilage nodules that exhibit the morphological, ultrastructural and biochemical characteristics of chondrocytes formed in vivo, has been achieved by differentiation of functional chondrocytes in culture. Recently, many attempts have been made to induce in vitro differentiation of ESCs into chondrogenic lineages. It has been reported that chondrogenic differentiation of ESCs was induced by various chondrogenic supplements when added during embryoid body (EB) differentiation. Such chondrogenic supplements include BMP-2 and BMP-4 (Kramer et al. Embryonic stem cell-derived chondrogenic differentiation in vitro: activation by BMP-2 and BMP-4 Mech. Dev. 92: 193-205, 2000), TGF-b3 (Kawaguchi et al. Osteogenic and chondrogenic differentiation of embryonic stem cells in response to specific growth factors Bone 36: 758-769, 2005), dexamethasone (Tanaka et al. Chondrogenic differentiation of murine embryonic stem cells: effects of culture conditions and dexamethasone J. Cell Biochem. 93: 454-462, 2004). As a different approach, it has been reported that macroscopic cartilage formation was achieved in EB culture derived from FACS sorted-mesodermal progenitor cells by supplying IGF-I, TGF-b3, BMP-4 and PDGF (Nakayama et al. Macroscopic cartilage formation with embryonic stem-cell-derived mesodermal progenitor cells J. Cell Sci. 116: 2015-2028, 2003). However, in spite of extensive successful approaches for chondrogenic differentiation of ESCs, these established methods require the formation of EBs. Chondrogenesis from ESCs has been performed in 2-D culture systems. To use ESCs for cartilage tissue engineering, it is imperative to develop well-defined and efficient protocols for directing differentiation to chondrogenic lineages in vitro in 3-D culture systems that are integrated and do not involve operator decisions.

Static cultures, such as the 2-D methods traditionally used for ES maintenance, culture and differentiation, suffer from several limitations such as the lack of mixing, poor control options, and the need for frequent feeding. Experiments in which cells are cultured in 2-D, wherein normal 3-D relationships with the extracellular matrix and other cells are distorted, may result in atypical cell behavior and thus produce mistaken conclusions. Stirred suspension culture systems offer attractive advantages of scalability and relative simplicity that may influence the viability and turnover of specific stages and types of stem cells (14). However, in stirred cultures of suspended cells, cell damage may result due to agitation and shear forces caused by the stirring. Processes using bioreactors to culture cells are being developed to provide dynamic cultivation systems, with controlled culture conditions, that will enable the expansion of cells in a 3-D environment. Analyzing cell interactions in more natural 3-D settings promises to provide conditions closer to those in living organisms (15;16). The use of bioreactors for hESC culture has been documented and provided some preliminary evidence that dynamic, 3-D conditions may provide a suitable environment to culture ES cells to form embryoid bodies (17).

Chang et al (8) pioneered bioencapsulation in the 1960's and Lim et al (19) eventually encapsulated xenograft islet cells for implantation into rats to correct diabetes. The use of alginate encapsulation has been mainly restricted to adult cells. Magyar et al (20) encapsulated murine ES cells in 1.1% alginate microbeads and cultured in 2-D on tissue culture plates, i.e. in static cultures. This resulted in the formation of “discoid” colonies, which further differentiated within the beads to give cystic EBs and later to EBs containing spontaneously beating areas. When Magyar et al. encapsulated ESC into 1.6% alginate microbeads and cultured in 3-D, differentiation was found to be inhibited at the morula-like stage, so that no cystic EB could be formed within the beads; although, when the ES cell colonies were released from the beads and cultured in 2-D, they were able to further differentiate into cystic EB with beating cardiomyocytes. The encapsulation of murine ES cells in alginate beads to generate EBs from InES cells has been attempted, but failed to yield sufficient chondrogenic differentiation (21). Mesenchymal stem cells (MSCs) encapsulated in alginate beads have been cultured in 3-D by placing the cell beads in static flask cultures and overlaying with growth medium to achieve chondrogenic differentiation yielding hyaline cartilage, although the proliferative capacity of the MSCs was found to be inhibited in alginate culture (22). Furthermore, chondrogenic differentiation has been demonstrated in 3-D culture using human adipose-derived adult stem (ADAS) cells seeded in alginate or agarose hydrogels, and in porous gelatin scaffolds (Surgifoam) (32).

Large scale production of differentiated cells from stem cells requires the integration of the various steps in ES culture. Current methods to form differentiated cells and tissues from pluripotent cells, such as ES cells, are fragmented, labor intensive and require a high level of training, which inevitably introduces operator to operator variability; also, such methods are performed in 2-D cultures, which do not simulate the 3-D environment that exists in vivo. This is unsatisfactory for clinical applications as current methods of maintenance culture and of differentiation cannot produce clinically relevant cell numbers.

Therefore, there exists a need for improved methods for stem cell culture, for expansion, and for integrated expansion and differentiation of stem cells, e.g. embryonic stem cells. Such methods are necessary for efficient maintenance growth and differentiation of undifferentiated pluripotent cells and for further differentiation of partially differentiated multipotent cells of the ectoderm, mesoderm and endoderm lineages. For clinical bone tissue engineering applications, there is a need for methods to achieve formation of “bone nodules” (bone-like tissue) or other tissue types. According to the present invention, this can be achieved in 3-D culture, using a single cell or a plurality of cells encapsulated in a support matrix.

The culture of a single cell, or clone, and the subsequent expansion and differentiation of the single clone is termed “clonality”. Clonally-derived ES cells have been shown to differentiate in vivo when implanted into mice, but to date, attempts to culture single undifferentiated ES cells in vitro have proved to be unsuccessful (23;24). In these reported studies, the single cell cultures were performed in 2-D and the cells were not terminally differentiated to mature cells.

Currently, no methods are available for screening the effects of the cell culture environment on individual pluripotent or multipotent cells. There is thus a desire for methods of identifying the effect of cell culture conditions, media and test compounds (such as synthetic chemical entities or naturally derived materials e.g. conditioned media, growth factors) on individual cells. Furthermore, the ability to perform a large number of such screening experiments simultaneously would allow the mass screening of a great number of process variables (chemicals, concentrations, combinations).

Citation or identification of any document in this application is not an admission that such document is available as prior art to the present invention.

### SUMMARY OF THE INVENTION

In one embodiment, the invention may be a method of cell culture comprising:
(a) providing a human embryonic stem (ES) cell encapsulated within a support matrix to form a support matrix structure, and,
(b) maintaining culture by maintaining the encapsulated cell in 3-D culture in maintenance medium.

The maintenance culture may be carried out in the absence of feeder cells and in the absence of feeder cell conditioned medium. Also the method may further comprise differentiating the encapsulated cell in 3-D culture in differentiation medium in conditions suitable for cell differentiation. The maintaining and differentiating stages may be performed in the same vessel.

A stimulus for differentiation may be provided, and this stimulus may comprise a stimulus for embryoid body formation, e.g., removal of, or reduced, exposure to a substance that suppresses differentiation; and/or, addition of, or increased, exposure to a substance that promotes embryoid body formation. The stimulus for differentiation may be a stimulus to an ectodermal, endodermal or, in particular, mesodermal lineage. The stimulus may also be for osteogenic or chondrogenic differentiation.

In an alternative embodiment, the invention may be a method of cell culture comprising:
(a) providing a single ES cell or a plurality of ES cells encapsulated within a support matrix to form a support matrix structure,
(b) maintenance culture by maintaining the encapsulated cell(s) in 3-D culture in maintenance medium, in conditions suitable for ES cell maintenance,
(c) osteogenic differentiation by differentiating the encapsulated cells in 3-D culture in differentiation medium, in conditions suitable for osteogenic differentiation.

Osteogenic differentiation of the encapsulated cells may comprise (i) incubating the encapsulated ES cells in 3-D culture in differentiation medium and providing a stimulus for embryoid body formation, then, (ii) incubating the encapsulated cells generated in (i) in differentiation medium and providing a stimulus for osteogenic differentiation.

Osteogenic differentiation of the encapsulated cells may alternatively comprise (i) incubating the encapsulated ES cells in 3-D culture in differentiation medium, then, (ii) incubating the encapsulated cells generated in (i) in differentiation medium and providing a stimulus for osteogenic differentiation.

Further, osteogenic differentiation of the encapsulated cells may comprise incubating the encapsulated ES cells in differentiation medium and providing a stimulus for osteogenic differentiation.

The ES cells may be of human, non-human primate, equine, canine, bovine, porcine, caprice, ovine, piscine, rodent, murine, or avian origin.

In one embodiment, a plurality of cells may be provided encapsulated within each support matrix structure. In another embodiment, a single cell may be provided encapsulated within each support matrix structure.

In the step involving the provision of a single ES cell or a plurality of ES cells encapsulated within a support matrix to form a support matrix structure, a plurality of support matrix structures may be provided.

The support matrix structure of the preceding embodiments may be in the form of a bead.

A human ES cell encapsulated within a support matrix may be used for assessing the effect of a test stimulus on cell maintenance and/or differentiation. Alternatively, the human ES cell encapsulated within a support matrix may be used for assessing the effect of culture media and/or conditions on cell maintenance and/or differentiation.

In one embodiment, the invention may be a method of cell culture comprising providing a human embryonic stem (ES) cell encapsulated within a support matrix to form a support matrix structure, maintaining culture by maintaining the encapsulated cell in 3-D culture in maintenance medium, and further comprising incubating the encapsulated cell in maintenance medium in the presence of a test compound and assessing the effect of the test compound on cell maintenance and/or differentiation.

In another embodiment, the invention may a method of cell culture comprising providing a human embryonic stem (ES) cell encapsulated within a support matrix to form a support matrix structure, maintaining culture by maintaining the encapsulated cell in 3-D culture in maintenance medium, and further comprising incubating the encapsulated cell in the presence of a test stimulus, in medium and conditions suitable for cell maintenance and/or differentiation and assessing the effect of the test stimulus on cell differentiation.

In yet another embodiment, the invention may be a method of cell culture comprising providing a human embryonic stem (ES) cell encapsulated within a support matrix to form a support matrix structure, maintaining culture by maintaining the encapsulated cell in 3-D culture in maintenance medium, and further comprising incubating the encapsulated cell in the presence of a test medium and/or test conditions and assessing the effect of the test medium and/or test conditions, on maintenance and/or differentiation of the cell. The cell may be provided with a test stimulus and the effect of test stimulus on maintenance and/or differentiation of the cell may be assessed.

In any of the preceding methods, in the step of providing a human embryonic stem (ES) cell encapsulated within a support matrix to form a support matrix structure, a plurality of cells may be encapsulated within each support matrix structure. Alternatively, a single cell is encapsulated within each support matrix structure.

The encapsulated cells may be provided in an array of culture vessels. The array of culture vessels may be a multi well or multi tube array. In any of the preceding methods, in the step of providing a human embryonic stem (ES) cell encapsulated within a support matrix to form a support matrix structure, a plurality of encapsulated cells may be provided in each culture vessel. Alternatively, a plurality of support matrix structures may be provided in each culture vessel. In another embodiment, a single encapsulated cell may be present in each culture vessel.

In one embodiment, the support matrix structure may be in the form of a bead. In another embodiment, the support matrix may consist of or comprise a hydrogel. In yet another embodiment, the support matrix may consist of or comprise alginate. Alternatively, the support matrix may further comprise gelatin. In another alternative embodiment, the support matrix may further comprise one or more material selected from the group comprising: gelatin, laminin, Bioglass™, hydroxyapatite, extracellular matrix, an extracellular matrix protein, a growth factor; an extract from another cell culture, an extract from an osteoblastic culture.

In one embodiment, the effect on cell maintenance and/or differentiation may be assessed by one or more method selected from the group consisting of: microscopic examination, detection of a stage-specific antigen or antigens and detection of gene expression.

The preceding embodiments may further comprise freezing the encapsulated cells or, alternatively, liberation of a cell or cells from the support matrix.

In one embodiment, the invention may be a cell or cells, or encapsulated cell or cells, obtained by the preceding embodiments.

In another embodiment, the invention may be encapsulated human ES cell or cells obtained by the method comprising providing a human embryonic stem (ES) cell encapsulated within a support matrix to form a support matrix structure, and maintaining culture by maintaining the encapsulated cell in 3-D culture in maintenance medium.

The invention may also be an encapsulated multipotent cell or cells, or an encapsulated osteogenic, chondrogenic or cardiomyogenic cell or cells, or an encapsulated terminally differentiated cell or cells, obtainable or obtained by the preceding embodiments.

In one embodiment, the cell or encapsulated cell can be used as a medicament.

In another embodiment, encapsulated osteogenic cell or cells can be used as a medicament for the treatment of a disease or condition requiring bone reconstruction. Alternatively, encapsulated osteogenic cell or cells can be used as a medicament for the treatment of a disease or condition selected from osteoporosis, bone breaks, bone fractures, bone cancer, osteocarcinoma, osteogenesis imperfecta, Paget's disease, fibrous dysplasia, bone disorders associated with hearing loss, hypophosphatasia, myeloma bone disease, osteopetrosis, over-use injury to bone, sports injury to bone, periodontal (gum) disease, and reconstructive surgery such as therapeutic maxifacial surgery, or cosmetic surgery. In yet another embodiment, encapsulated osteogenic cell or cells may be used in the manufacture of a medicament for the treatment of a disease or condition requiring bone reconstruction. In yet another embodiment, the encapsulated osteogenic cell or cells may be used in the manufacture of a medicament for the treatment of a disease or condition selected from osteoporosis; bone breaks, bone fractures, bone cancer, osteocarcinoma, osteogenesis imperfecta, Paget's disease, fibrous dysplasia, bone disorders associated with hearing loss, hypophosphatasia, myeloma bone disease, osteopetrosis, over-use injury to bone, sports injury to bone, and periodontal (gum) disease.

In another embodiment, encapsulated chondrogenic cell or cells may be used as a medicament for the treatment of a disease or condition selected from arthritis, a cartilage disease or disorder, cartilage repair, cosmetic reconstructive surgery; rheumatoid and osteoarthritis. Alternatively, encapsulated chondrogenic cell or cells may be used in the manufacture of a medicament for the treatment of a disease or condition selected from arthritis, a cartilage disease or disorder, cartilage repair, reconstructive surgery; cosmetic reconstructive surgery, rheumatoid and osteoarthritis.

In one embodiment, the invention may be a method of treatment of a subject comprising administration of encapsulated cells to a subject. In another embodiment, the invention may be a method of treatment of a disease or condition requiring bone reconstruction comprising administration of encapsulated osteogenic cell or cells to a subject. Alternatively, the invention may be a method of treatment a disease or condition selected from osteoporosis; bone breaks, bone fractures; bone cancer, osteocarcinoma, osteogenesis imperfecta, Paget's disease, fibrous dysplasia, bone disorders associated with hearing loss, hypophosphatasia, myeloma bone disease, osteopetrosis; over-use injury to bone, sports injury to bone, and periodontal (gum) disease comprising administration of encapsulated osteogenic cell or cells. In yet another embodiment, the invention may be a method of treatment of a disease or condition selected from arthritis, a cartilage disease or disorder, cartilage repair, rheumatoid and osteoarthritis comprising administration of encapsulated cell or cells to a subject.

In one embodiment, the invention may be a method of reconstructive surgery selected from therapeutic or cosmetic surgery comprising administration of encapsulated cell or cells to a subject. In another embodiment, the invention may be a method of reconstructive surgery selected from therapeutic or cosmetic surgery comprising administration of encapsulated osteogenic cell or cells or chondrogenic cell or cells to a subject.

In one embodiment, the invention may be a pharmaceutical composition comprising encapsulated cell or cells and a pharmaceutically acceptable carrier or diluent. In another embodiment, the pharmaceutical composition may be formulated for administration by injection, or by endoscopy.

In one embodiment, the invention may be a bone or cartilage tissue derived from encapsulated cell or cells. Alternatively, the invention may be a cell scaffold having seeded on or impregnated therein encapsulated cells. In another embodiment, the invention may be a pre-filled administration device, such as a syringe, containing a pharmaceutical composition comprising encapsulated cell or cells and a pharmaceutically acceptable carrier or diluent, and may alternatively be formulated for administration by injection, or by endoscopy

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. 112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawing, in which:

FIGS. 1 and 2 show immunofluorescence stained with antibody for Oct4. 130 day paraffin embedded/sectioned hESC aggregates revealed positive immunostaining for Oct-4. (Inset—negative and positive control)

FIGS. 3 and 4 show immunofluorescence stained with anti-TRA-1-81. Immunostaining of paraffin embedded/sectioned 130 day hESC aggregates exhibited strong immunoreactivity to this antibody indicating retention of pluripotency. (Inset—negative and positive control)

FIGS. 5 and 6 show immunofluorescence stained with anti-SSEA-4. Undifferentiated HESC aggregates, revealed positive immunostaining for SSEA-4 antibody. (Inset—negative and positive control)

FIG. 7 shows RT-PCR Analysis; RT-PCR analysis shows expression of pluripotent markers. Oct4 and Nanog in both 175 day and 260 days hES cell aggregates. Lane A is 175 day old hES cell aggregates, lane B 260 day old hES cell aggregates, lane C is a negative control. GAPDH expression was used as an internal control.

FIG. 8 shows growth of a single mES cell encapsulated within a hydrogel 1.1% w/v alginate, 0.1% v/v gelatin bead for 10 days in static 3-D culture in M2 medium. Scale bars are 50 μm. The single ES cell undergoes division and a small colony of cells is formed at around 10 days.

FIG. 9 shows a schematic diagram of the integrated maintenance and osteogenic differentiation strategy. The steps were:
a) encapsulation of undifferentiated mESCs in alginate plus gelatin microbeads and introduction into a 3-D bioreactor;
b) culture for 3 days in maintenance medium (M2) to increase mES cell numbers and form suitable cell clusters to allow the formation of 3D multiprogenitors;
c) culture for 5 days in EB formation medium (M1);
d) culture for 21 days in osteogenic medium (Buttery) to allow osteoinduction and 3-D bone formation.

FIG. 10 shows tissue morphology in the alginate beads. The alginate beads retain their spherical shape and cell clustering becomes evident: (a) day 3 (scale bar length=1000 μm); (b) day 7 (scale bar length=500 μm); (c) day 21 (scale bar length=500 μm). Hematoxylin/eosin stained thin-sections of the hydrogels at various times showing tissue development: (d) day 3 (scale bar length=20 μm); (e) day 8 (scale bar length=20 μM); (f) day 22 (scale bar length=20 μm).

FIG. 11 shows cell viability (inset) within the alginate beads as demonstrated by live/dead staining (green indicates live and red indicates dead cells; scale bar length=100 μm). The biochemical performance per bead in the 3D cultures was assessed by employing the MTS assay for metabolic activity (▴; n=24) and the alkaline phosphatase assay (; n=6) and alizarin red quantification (▪; n=6) for mineralized tissue formation. Error bars represent the standard error. */# significant increase/decrease (p<0.05).

FIG. 12 shows characterization of the encapsulated mnESCs. Immunocyto-chemistry confirms the maintenance of the undifferentiated state at day 3: (a) DAPI (blue) and CD9 (red), (b) DAPI (blue), (c) Oct-4 (green). When the 3D cultures were grown in EB formation medium (days 3-8), generation of mesodermal tissue became evident at day 8: (d) DAPI (blue) and Flk-1 (green). Insets represent the negative controls obtained from mESCs cultured on tissue culture plastic (2D). Scale bar length=20 μm.

FIG. 13 shows mineralized tissue formation characterization. (a) Balb/c mouse bone alizarin red S positive control and (b) Balb/c mouse von Kossa positive control. Mineralized tissue formation in the alginate beads on day 22 was demonstrated by (c) alizarin red S and (d) von Kossa staining. Hematoxylin/eosin staining of the midsection of the alginate bead revealed the formation of tissue in the core of the hydrogels at day 29 (e-f). Examination of the same sections for bone formation at day 29 showed a more pronounced staining for alizarin red S (g) and von Kossa (h). Immunocytochemistry at day 29 confirmed the presence of terminally differentiated osteoblasts: (i) day 29 section stained with DAPI (blue) and immunostained for osteocalcin (green) and the inset (j) shows Balb/c mouse bone negative control stained in the same way; (k) day 29 section stained for DAPI (blue) and immunostained for osteocalcin (green) at higher magnification and the inset (l) shows Balb/c mouse bone positive control; (m) day 29 section stained with DAPI (blue) and immunostained for OB-cadherin (green) and the insets show (n) Balb/c mouse bone positive control and (o) Balb/c mouse bone negative control; (p) day 29 section stained with DAPI (blue) and immunostained for collagen-I (green) and the insets show (q) Balb/c mouse bone positive control and (r) Balb/c mouse bone negative control. Scale bar length for (a-f) is 100 μm and for (g-j) is 20 μM.

FIG. 14 shows gene expression analysis of osteogenic markers during the bone formation period at days 15 (d15), 22 (d22), and 29 (d29). L=100 bp DNA ladder. RT-ve=RT-negative control in the absence of reverse transcriptase enzyme at day 29 with GapDH primers. −ve=PCR negative control using water instead of template with GapDH primers. +ve=positive control using MC-3T3-E1 cells cultured for 10 days in osteogenic medium.

FIG. 15 shows evaluation of tissue mineralization using micro-computed tomography (micro-CT). The alginate beads were evaluated at day 29 for the extent of mineralization of the bone aggregates. (a-b) False color, 3D sector reconstruction at day 29 of a single alginate bead selected at random. The inset represents the false color positive control using a Balb/c mouse femur. Coloration in false color images indicates the level of attenuation from the highest (yellow) to purple and to the lowest (black) indicating hard to soft tissue, respectively. (c) shows a grayscale transmission image at day 29 of an alginate bead (the red arrow indicates soft tissue surrounding a mineralized aggregate). The inset shows a negative control grayscale transmission image using an alginate bead without any cells (dotted line denotes bead border). (d) False color, 2D cross section of a day 29 alginate bead. Scale bar length=100 μm.

### DETAILED DESCRIPTION

The invention provides a method of cell culture comprising:
(a) providing a human embryonic stem (ES) cell encapsulated within a support matrix to form a support matrix structure, and,
(b) maintaining culture by maintaining the encapsulated cell in 3-D culture in maintenance medium.

In culture methods of the invention the ES cell may be provided as multiple individual cells and/or aggregates of cells encapsulated within the support matrix structure, or as a single cell encapsulated within the support matrix structure for clonal expansion.

The choice of maintenance medium for maintenance growth of the cells to increase numbers of cells within the support matrix structure (i.e. expansion, in which the cells undergo self-renewal by cell division) may depend upon the type of cells employed and their requirements for growth. Any media that supports cell growth, ideally with minimal or no cell differentiation, may be suitable for use as a maintenance medium in methods of the invention. Various appropriate maintenance media are known in the art.

In a preferred embodiment, maintenance culture may not involve exposure to feeder cells, conditioned media, or human or animal cell extracts in the maintenance medium; thus, maintenance culture may be carried out in the absence of feeder cells and in the absence of feeder cell conditioned medium.

Current methods of culturing hES cells require either the use of feeder cells to support the maintenance of the hES cells in an undifferentiated state or the use of conditioned culture medium (1). Also, in current methods the cells require regular passaging to remove those hES cells that have spontaneously differentiated. Furthermore, the culture conditions may require products derived from animals which carry a risk of disease transfer if the resultant hES cells are to be used as a clinical therapeutic. Researchers are striving to develop methods for the maintenance and expansion of hES cells which are amenable to large scale production to supply sufficient numbers of hES cells or their differentiated derivatives for therapeutic applications.

To this end the present inventors have developed a surprisingly simple process which appears to replicate the physical environment of the early pre-implantation embryo and which enables the long-term culture of encapsulated hES cells in their undifferentiated state, without the need for passaging. Surprisingly the inventors have found that hES cells can be maintained undifferentiated using the methods of the current invention in the absence of feeder cells, in unconditioned media, for periods of up to 130 days. The inventors hypothesize that the physical environment provided by support matrices that encapsulate the hES in methods according to the present invention negates the requirement for feeder cell support or exposure to conditioned medium. The methods of the present invention are amenable to standardization, regulation, and production scale-up for production of hES cells for therapeutic applications.

Suitable maintenance medium for human ES cells include DMEM/F12 medium supplemented with 20% v/v KNOCKOUT™ SR, 2 mM L-glutamine, 0.1 mM non-essential amino acids solution (all from Gibco Invitrogen, Life Technologies, Paisley, UK), 0.1 mM 2-mercaptoethanol (2ME) (Sigma-Aldrich, Dorset, UK) and 4 ng/ml human recombinant basic fibroblast growth factor (bFGF, FGF-2) (157 aa) (R&D Systems, Oxon, UK). VitroHES™ (Vitrolife AB, Kungsbacka, Sweden, http://www.vitrolife.com) supplemented with 4 ng/ml human recombinant basic fibroblast growth factor (hrbFGF) is also a suitable medium in which to culture hES cells. Both of these media are usually used with feeder cells; however in culture methods of the invention in which cells are encapsulated, these media can be used without concomitant use of feeder layers. Feeder free culture of unencapsulated hES cells is possible with conditioned medium and additional growth factors. However, Xu et al (2005) (25) have shown that unconditioned media containing KNOCKOUT™ SR activates BMP signaling activity in unencapsulated hES cells to a greater extent than MEF conditioned medium. Therefore a defined medium for feeder free maintenance of unencapsulated hES cells is presently unavailable.

Maintenance of unencapsulated hES cells in a feeder free environment using specific cell signaling molecules has previously been achieved only for relatively short periods of time (Sato et al. Nat. Med., 10: 55-63, 2004). Surprisingly, in the methods of the current invention, specific signaling molecules are not required to maintain the hES cells in an undifferentiated state. Nevertheless, as such studies continue to identify molecules which improve the maintenance and expansion of hES cells in an undifferentiated state, they can be used in the methods of the current invention to further enhance the in vitro environment for encapsulated hES cell culture.

In methods of the invention, encapsulated ES cells can be grown in unconditioned media. The various media and details of the combinations of growth factors currently used for maintenance of unencapsulated hES cells are reviewed in (1). These media can be used or adapted for use in methods of the invention, without feeder cells and without the need for the medium to be conditioned.

In a preferred aspect, the invention provides a method of cell culture comprising:
(a) providing a human ES cell encapsulated within a support matrix to form a support matrix structure,
(b) maintaining culture by maintaining the encapsulated cell in 3-D culture in maintenance medium in conditions suitable for cell maintenance, then,
(c) differentiating the encapsulated cell in 3-D culture in differentiation medium in conditions suitable for cell differentiation.

The choice of differentiation medium for differentiation of the pluripotent hES cells may depend upon the type of cells employed, their requirements for growth and the stimulus required for differentiation. Any media that will support differentiation may be suitable for use as a differentiation medium in methods of the invention. In practice, differentiation media can be similar in composition to maintenance media, but the differentiation media will not contain a substance or substances included in the maintenance medium to suppress differentiation. Suitable differentiation media for hES cells include medium [Alpha-Modified Eagles Medium (αMEM), 10% (v/v) fetal calf serum, 100 units/mL penicillin and 100 μg/mL streptomycin]. Differentiation media may be generated by addition of a stimulus for differentiation, such as a growth factor, to maintenance media.

Conditions suitable for maintenance and/or differentiation of encapsulated pluripotent or encapsulated multipotent cells in 3-D culture may include standard culture conditions for the cell type used, e.g. for ES cell culture, suitable conditions would include the use of ES maintenance and/or differentiation culture media and environmental conditions such as 37° C. and 5% CO₂.

Using methods of the invention for maintenance (expansion) and/or differentiation, colony or tissue formation is performed in 3-D culture, which may be static e.g. in a tissue culture plate, or in suspension, e.g. in a flask or bioreactor. In 3-D culture, organized structures and greater numbers of cells can be formed as the conditions more closely correspond to physical environment in an in vivo situation. In 3-D culture, the cells grow in three-dimensions.

Appropriate 3-D suspension culture conditions for performing cell culture methods of the invention can be achieved using a low shear, high mixing, “dynamic” environment. This enables sufficient nutrients and gases to permeate the support matrix structure employed. Suitable bioreactor systems to provide a low shear, high mixing, dynamic environment for 3-D culture include the NASA HARV bioreactor (Synthecon, USA), European Space Agency bioreactor (Fokker, Netherlands), RWV Bioreactor (Synthecon, USA) or other simulated microgravity or perfused systems such as airlift bioreactors. For methods involving osteogenic differentiation, the NASA HARV bioreactor is suitable.

Suitable methods of maintenance and differentiation may be performed as integrated methods, in which the maintenance and differentiation steps are performed sequentially in a single, i.e. the same, vessel. Integrated methods of methods of maintenance and differentiation may be suitably performed in suspension culture in a flask or bioreactor. In the maintenance growth phase, the encapsulated pluripotent ES cell(s) divide, and cell numbers may be increased so that colonies of cells form within the support matrix structure. The encapsulated cells may then be differentiated, forming further differentiated or terminally differentiated cells, all within the 3-D matrix structure. In methods of the invention, the further differentiated or terminally differentiated cells can then be maintained, allowing the cells to divide so that cell numbers are increased and colonies of cells form within the support matrix structure.

The use of a fully-integrated process may enable the sequential change from expansion of undifferentiated cells through the timed and controlled differentiation triggered by the addition or subtraction of key cell signaling molecules in the culture media. The reduced cell-handling requirements using the methods of the invention can limit the exposure of the cells to potential contaminants and environments which may impact cell viability. In addition, monitoring of the cell culture conditions in a real-time manner may enable the development of the standards required for clinical products.

Some cell lines may undergo spontaneous differentiation after cycles of cell division in maintenance growth, particularly if the conditions are such that differentiation is not suppressed. Conditions suitable for cell differentiation may comprise a stimulus for differentiation of the pluripotent ES cell to a multipotent cell. The stimulus for differentiation of an ES cell to a multipotent cell can be a stimulus for embryoid body formation, for example removal of, or reduced, exposure to a substance that suppresses differentiation; and/or addition of, or increased, exposure to a substance that promotes embryoid body formation. The conditions suitable for cell differentiation may comprise a stimulus for further differentiation of a multipotent cell; e.g. which can be provided before, at the same time, or after the stimulus for differentiation of the ES cell. Methods of the invention involving differentiation may be performed without provision of a stimulus for embryoid body formation; instead the conditions suitable for differentiation may simply comprise a stimulus for differentiation, e.g. to an ectodermal, endodermal or mesodermal linage.

The stimulus for differentiation can be a stimulus for differentiation to an ectodermal, endodermal or mesodermal linage. Suitable stimuli are known in the art as listed below, and are discussed, for example in reference (1).

Preferably the stimulus for differentiation may be a stimulus for differentiation into a mesodermal skeletal lineage cell, e.g. a stimulus for osteogenic or chondrogenic differentiation.

The stimulus for osteogenic differentiation can be a supplement provided to the culture medium, e.g. one or more of ascorbic acid, β-glycerophosphosphate or dexamethosone.

The stimulus for chondrogenic differentiation can be a supplement provided to the culture medium, e.g. monothioglycerol (MTG) and IGF-1, TGF β1, BMP 2 or BMP 4.

The duration of the maintenance and differentiation steps may depend on the type of cells cultured and the aim of the cell culture. The inventors have demonstrated that using a method of the present invention, encapsulated human ES cells can be maintained, undifferentiated, for 130 days in the absence of feeder cells or conditioned medium conventionally used to maintain pluripotency. In maintenance cultures, it may be desirable to culture the encapsulated hES cells for periods of up to 130 days or longer, if desired, to provide increased numbers of undifferentiated cells. Hence the invention provides methods that can be used for long term maintenance culture of encapsulated hES cells, e.g. for periods over 8 days, e.g. for about 14, 21, 28, 35, 42, 49, 56 days, up to 130 days and beyond.

In integrated maintenance and differentiation methods, initial maintenance culture of encapsulated cells in step (b), i.e., maintaining culture by maintaining the encapsulated cell in 3-D culture in maintenance medium in conditions suitable for cell maintenance, should be of sufficient length to permit formation of cell clusters, e.g. from 1 to 6 days, preferably from 2 to 5 days, most preferably 3 or 4 days. Differentiation culture can be for up to 40 days. Some culture methods of the invention may involve an initial differentiation period in the presence of a stimulus for EB formation, followed by a further differentiation period in the presence of a stimulus for differentiation of multipotent cells into more differentiated cell lineages e.g. into osteoblasts or chondrocytes. Suitably the initial differentiation period may be from 3 to 7 days, preferably from 4 to 6 days most preferably about 5 days. When further differentiation is performed, the further differentiation period, will generally be from 14 to 28 days, suitably about 20 to 22 days, e.g. 21 days.

For osteogenic differentiation of encapsulated ES cells according to a method of the invention, the initial maintenance period is typically 2 to 4 days, e.g. 3 days; the initial differentiation period may be 4 to 6 days, e.g. 5 days; and the further differentiation period may be 14 to 28 days, e.g. 20, 21 or 22 days; these culture times are generally suitable to achieve osteoinduction and 3-D bone formation.

Using methods of the invention that include a differentiation phase, encapsulated multipotent cells can be differentiated to more differentiated cells, such as terminally differentiated cells. Differentiation of multipotent cells to more, or terminally, differentiated cells may be suitably achieved using conditions for cell differentiation which comprise a stimulus for further differentiation of the multipotent cell.

Methods of the invention can also be used for in vitro maintenance and/or differentiation of single cells encapsulated within a support matrix, e.g. to provide homogeneous colonies or tissues. Thus, in some embodiments of methods of the invention, in step (a), i.e., providing a human ES cell encapsulated within a support matrix to form a support matrix structure, the support matrix structures are such that a single ES cell may be encapsulated within a support matrix to form a support matrix structure.

An ES cell can be encapsulated into a support matrix to provide a support matrix structure, such as a bead, containing a single cell. The encapsulated single cell can then be grown into cell colonies, optionally EB structures can be formed, and the partially differentiated cells can eventually be differentiated into the desired cell lineage. This is useful for obtaining a clonally derived cell population for providing a pure homogeneous cell population for clinical use. Also, this is useful for screening purposes as it permits examination of 3-D embryoid body formation, cell division of ES cells, or investigation of the influences of the microenvironment on a single pluripotent cell. Differentiation of a single ES into the differentiated mature cell types can also be investigated, thus demonstrating the in vitro pluripotency potential of ES cells.

Alternatively, in step (a), i.e., providing a human ES cell encapsulated within a support matrix to form a support matrix structure, a plurality of cells are provided encapsulated within a support matrix structure. These may be present as multiple single cells, or cell aggregates (i.e. clumps/colonies) or a mixture thereof. These aspects are particularly useful for generation of large quantities of differentiated cells, e.g. for tissue engineering applications, for research, or for clinical use, but can also be used for screening purposes.

Generally, in cell culture methods of the invention, in step (a) a plurality of support matrix structures may be provided.

The invention provides integrated 3-D culture methods for ES maintenance, optional EB formation, and differentiation. Mesodermal cells derived from the ES can be differentiated into cardiomyogenic, chondrogenic or osteogenic cells under the influence of cardiomyogenic, chondrogenic or osteogenic stimuli respectively.

Using methods of the invention, osteogenic differentiation can be achieved in 3-D culture resulting in the formation of “bone nodules” (bone-like tissue), or other tissue types for clinical bone tissue engineering applications can be achieved in 3-D culture. Methods of the invention can be adapted for automation of the culture system, to provide low maintenance, high efficiency systems for generation of differentiated cells. For example, these methods can be used for production of cardiomyogenic, chondrogenic or osteogenic cells from mES cells or hES (human embryonic stem) cells.

Thus, in alternative embodiments, culture methods of the invention are particularly useful for osteogenic differentiation of ES cells, and a particularly preferred method of cell culture may comprise:
(a) providing a single ES cell or a plurality of ES cells encapsulated within a support matrix to form a support matrix structure,
(b) maintaining the encapsulated cell(s) in 3-D culture in maintenance medium, in conditions suitable for ES cell maintenance,
(c) osteogenic differentiation by differentiating the encapsulated cells in 3-D culture in differentiation medium, in conditions suitable for osteogenic differentiation.

The ES cells are preferably murine or human ES cells, however osteogenic differentiation methods of the invention are applicable to ES cells of human, non-human primate, equine, canine, bovine, porcine, caprice, ovine, piscine, rodent, murine, or avian origin.

Preferred support matrices comprise alginate, and those that comprise alginate and gelatin are particularly preferred. Support matrix structures are preferably in the form of beads. The method can be performed in static suspension culture, but preferably is performed in a low shear, high mixing dynamic environment, e.g. provided by a bioreactor, such as a NASA HARV bioreactor.

The maintenance media routinely used to culture the ES cells in 2-D may be suitable for use in this method, as are other media described above. Suitable conditions are 37° C., 5% CO₂. Maintenance culture is performed for 1 to 6 days, preferably 2 to 4 days, more preferably around 3 days.

Osteogenic differentiation of the encapsulated cells may be suitably performed by
(i) incubating the encapsulated ES cells in 3-D culture in differentiation medium and providing a stimulus for embryoid body formation, then,
(ii) incubating the encapsulated cells generated in (i) in differentiation medium and providing a stimulus for osteogenic differentiation.

The differentiation medium can be, for example, any medium routinely used for osteogenic differentiation of ES cells in 2-D culture. The differentiation media used in conditions suitable for embryoid body formation and for subsequent osteogenic differentiation can be different. For murine cells, the stimulus for embryoid body formation can be removal of exposure to LIF, or where the maintenance phase was performed as co-culture, removal of exposure to LIF secreting cells.

For osteogenic differentiation to form bone nodules, the incubation in step (i) is typically performed for about 1 to 6 days, preferably about 2 to 5 days, most preferably about 3 or 4 days and the incubation in step (ii) is typically performed for 21 to 28 days, preferably 20 to 22 days e.g. 21 days.

In differentiation methods of the invention the embryoid body formation step is not always necessary, and thereby in some embodiments exposure to a stimulus for embryoid body formation is omitted; in this aspect osteogenic differentiation of the encapsulated cells is suitably performed by
(i) incubating the encapsulated ES cells in 3-D culture in differentiation medium, then,
(ii) incubating the encapsulated cells generated in (i) in differentiation medium and providing a stimulus for osteogenic differentiation.

Suitably the ES cells are exposed to differentiation medium in step (i) for about 1 to 6 days, preferably about 2 to 5 days, most preferably about 3 or 4 days and following provision of a stimulus for osteogenic differentiation in step (ii) incubation is typically performed for 21 to 28 days, preferably 20 to 22 days e.g. 21 days.

Alternatively, osteogenic differentiation of the encapsulated cells may be performed by incubating the encapsulated cells in differentiation medium and providing a stimulus for osteogenic differentiation.

In this instance the cells may be incubated in differentiation medium in the presence of a stimulus for osteogenic differentiation for 21 to 28 days.

Known in vitro inducers of osteogenic differentiation can be used, preferably in step (ii), to further differentiate multipotent cells. Briefly, serum, ascorbate (ascorbic acid), or L-ascorbate-2-phosphate (a long acting ascorbate analogue), β-glycerophosphate, and dexamethasone are each known to act as in vitro inducers of osteogenic differentiation. In current techniques, serum, ascorbate, and dexamethasone are absolute requirements for nodule formation whereas β-glycerophosphate promotes or enhances mineralization (26). The only morphological feature specific to osteoblasts is located outside the cell, in the form of a mineralized extracellular matrix. Bone nodule formation in vitro is subdivided into three stages: (i) proliferation, (ii) ECM secretion/maturation and (iii) mineralization.

Methods of the invention can be operated on an industrial process scale for the production of specific differentiated cell types. For example, bone formation can be achieved starting with ES cells encapsulated in alginate or alginate-based beads and performing cultures in a bioreactor. This automated, integrated process is efficient, readily controlled and gives a significant reduction in the time taken to form bone tissues compared to prior art 2-D methods and 3-D methods.

Encapsulation of an ES cell or cells in a support matrix, e.g. to form beads, results in an environment conducive to the maintenance of the ES cells, to differentiation, optionally via EB formation, and further differentiation, e.g. osteogenic differentiation. Methods of the invention permit automation, control, optimization, and intensification of the process, enabling production of clinically relevant numbers of cells, such as osteogenic cells, required for clinical applications.

Osteogenic methods of the invention are applicable to pluripotent cells of any origin, for example, the pluripotent cell of human, non-human primate, equine, canine, bovine, porcine, caprice, ovine, piscine, rodent, murine, or avian origin.

Methods of the invention for maintenance of hES cells can be adapted to provide methods of screening to assess the effect of the cell environment (culture conditions, media, test stimuli, compounds) on maintenance growth and/or differentiation. Accordingly, the invention provides the use of a hES cell encapsulated within a support matrix for assessing the effect of a test compound or stimulus on cell maintenance and/or differentiation. The invention yet further provides use of a hES cell encapsulated within a support matrix for assessing the effect of culture media and/or conditions on cell maintenance and/or differentiation.

Also provided is a method of identifying a compound capable of modulating hES cell maintenance and/or differentiation comprising:
(a) providing a hES cell encapsulated within a support matrix to form a support matrix structure,
(b) incubating the encapsulated hES cell in maintenance medium in the presence of a test compound,
(c) assessing the effect of the test compound on hES cell maintenance and/or differentiation.

Using this screening method of the invention, it is possible to identify compounds that promote cell maintenance, by suppressing differentiation of the pluripotent or multipotent cells, and to identify compounds that promote differentiation. The test compound, or mixture of compounds, can be naturally produced or chemically synthesized.

Additionally, provided is method of identifying a stimulus capable of modulating hES cell differentiation comprising:
(a) providing a hES cell encapsulated within a support matrix to form a support matrix structure,
(b) incubating the encapsulated hES cell in the presence of a test stimulus, in medium and conditions suitable for cell maintenance and/or differentiation,
(c) assessing the effect of the test stimulus on hES cell differentiation.

Using this method of the invention, it is possible to identify stimuli, e.g. compounds and/or conditions, that suppress or promote differentiation.

In a further aspect, the invention provides a method of assessing the effect of culture media and/or conditions on hES cell maintenance and/or differentiation comprising:
(a) providing a hES cell encapsulated within a support matrix to form a support matrix structure,
(b) incubating the encapsulated hES cell in the presence of a test medium and/or test conditions,
(c) assessing the effect of the test medium and/or test conditions, on maintenance and/or differentiation of the hES cell.

This method is useful for optimization of culture conditions to enhance cell maintenance, suppress differentiation, or promote differentiation. In this method of assessment, optionally the cell can be incubated in the presence of a test compound/stimulus and the effect of the test compound/stimulus on maintenance and/or differentiation of the cell can be assessed.

Screening methods can be performed so that in step (a) a plurality of cells is encapsulated within each support matrix structure, or so that in step (a) a single cell is encapsulated within each support matrix structure.

In preferred screening methods of the invention, encapsulated single cells are used, e.g. in the form of a bead, where each bead contains a single cell, such as an ES cell. By culturing a bead containing a single cell individually, suitably in multiple-well plates (which may be in array format, e.g. multi-well plates, such as 96 well plates) or micro-bioreactors, it is possible to perform multiple screens contemporaneously, to evaluate and optimize culture medium and conditions, and to screen chemically synthesized compounds, various growth factors, extracellular matrix proteins etc., for the effects that they have on cell growth and differentiation.

Screening methods can be configured so that encapsulated cells are provided in an array of culture vessels, for example as a multi-well or multi-chamber array. Preferably, in step (a) a plurality of encapsulated cells is present in each culture vessel, which can be achieved by providing a single support matrix structure, e.g. a bead, containing a plurality of cells; or more preferably by providing in step (a) a plurality of support matrix structures in each culture vessel. In this second approach, each support matrix structure, e.g. bead, can contain a single cell or a plurality of cells. In alternative screening methods one encapsulated cell is present in each culture vessel.

The use of methods as described herein may allow the rapid culture of single hES cells in a controlled environment. This can enable high throughput screening of many different culture environments in parallel or of many different cell types in the same culture environment in parallel. Suitably, 5 to 20 beads each containing a single hES cell can be provided in a single culture vessel, e.g. a well of a multi-well plate. Each bead constitutes an individual growth environment since a single cell within a bead will not be in direct contact with the single cells encapsulated within neighboring beads. Placing multiple beads in a single well allows time study analyses to be performed, since each bead will be exposed to identical conditions. Culturing in multi-well plates enables screening for multiple conditions and facilitates statistical analysis of the results. The use of robotics can facilitate the automation of the process, e.g. by feeding the cultures. Encapsulation of single cells within the beads ensures that the individual cultures are not disturbed during feeding or other manipulations.

Screening methods of the invention can be performed in 2-D culture (static or suspension) in a culture vessel or in 3-D culture in a bioreactor, such as a HARV bioreactor. The use of micro-bioreactors which have micro-channels enables constant, perfused feeding of the 3-D cultures, facilitating even more elaborate screening experiments and automation. Screening methods of the invention can be performed in high throughput format.

For screening uses or methods according to the invention, the effect of a test compound, test stimulus, culture medium, and/or conditions on cell maintenance and/or differentiation can be assessed by one or more method selected from the group consisting of microscopic examination, detection of a stage-specific antigen or antigens, and detection of gene expression levels, e.g. by RT-PCR or using a DNA or RNA micro array.

The support matrix utilized for encapsulation can be permeable to allow diffusion and mass transfer of nutrients, metabolites, and growth factors. A cell or cells encapsulated within a support matrix can be provided in the form of a bead, e.g. a generally spherical bead. By “encapsulated” it is meant that the cell or cells are entirely embedded within the support matrix. The shape of the bead is not particularly relevant provided that the dimensions, e.g. surface area to volume ratio, are such that nutrients, metabolites, cytokines etc., can readily diffuse into/out of the bead to reach the cell or cells embedded within the bead.

It is particularly preferred that the support matrix structures, e.g. beads, are constructed of a support matrix material that remains intact during the culture time, which may be 3 to 4 months or longer for maintenance; or for up to 30 to 40 days, as is the case in osteogenic differentiation culture methods. The cell or cells encapsulated within the support matrix can be placed into an 3-D culture vessel such as a RWV bioreactor (Synthesis, USA) or other simulated microgravity or perfused bioreactor), and incubated in maintenance and/or differentiation medium without significant damage for prolonged periods.

Preferably the support matrix material consists of or comprises a hydrogen material, e.g. a gel-forming polysaccharide, such as an agarose or alginate, (typically in the range of from about 0.5 to about 2% w/v, preferably at from about 0.8 to about 1.5% w/v, more preferably about 0.9 to 1.2% v/v). The matrix may consist of alginate alone or may comprise further constituents such gelatin (typically at from about 0.05 to about 1% w/v, preferably at from about 0.08 to about 0.5% v/v). The inclusion of gelatin assists in production of a uniform bead size and helps to maintain structural integrity. This is important because alginate hydro gels lose Ca²⁺ cations after prolonged culture, which weakens the structural integrity of the beads. Inclusion of gelatin in alginate support matrix beads enables cell-mediated contraction and packing of the scaffold material.

Alginate is a water-soluble linear polysaccharide extracted from brown seaweed and is composed of alternating blocks of 1-4 linked α-L-glucuronic and β-D-mannuronic acid residues. Alginate forms gels with most di- and multivalent cations, although Ca²⁺ is most widely used. Calcium cations take part in the interchain binding between G-blocks and give rise to a 3-dimensional network in the form of a gel. The binding zone between the G-blocks is often described as the “egg-box model” (27).

Alginate and alginate-based support matrices, suitably in the form of beads (e.g. alginate plus gelatin beads), have been found to be particularly appropriate for use in methods of the invention, as they maintain their integrity in the culture conditions employed.

The support matrices can be modified with a variety of signals (such as laminin, collagen, or growth factors) to enhance the desired cellular behavior. Thus, the support matrix may comprise one or more material selected from the group comprising: laminin, Bioglass™, hydroxyapatite, extracellular matrix, an extracellular matrix protein, a growth factor; an extract from another cell culture, and for osteogenic differentiation, an extract from an osteoblastic culture.

Extracellular matrix (ECM) has been used in 2-D culture as a stimulus to achieve osteogenic differentiation of ES cells (Hausemann & Pauken. Differentiation of embryonic stem cells to osteoblasts on extracellular matrix, 10th Annual Undergraduate research Poster Symposium, Arizona State University, 2003). Numerous growth factors are known in the art that stimulate differentiation of pluripotent stem cells such as ES cells, for example: bone morphogenesis protein 4 (BMP4), which enhances mesoderm formation and also bone formation (Nakayama et al. J Cell Sci 116(10): 2015, 2003); retinoic acid, which stimulates mesoderm formation; hedgehog proteins, such as sonic hedgehog which stimulates mesoderm to osteoprogenitor differentiation; and the bone morphogenesis proteins BMPs 1 to 3 and 5 to 9, which stimulate bone induction.

Calcium alginate or calcium alginate-based support matrices are favored for osteogenic culture and differentiation. Calcium ions are used as a chelating agent in formation of the beads and may provide a local source of calcium to aid osteogenic mineralization.

The use of alginate comprising gelatin as a support matrix material for encapsulation to form support matrix structures, e.g. to form beads, is particularly preferred in methods where single cells are encapsulated to form beads with a single cell per bead, and then cultured to form colonies.

Suitably, beads containing single cells are from about 20 to about 150 microns, preferably from about 40 to about 100 microns in diameter. Beads containing a plurality of cells are generally from about 2.0 to about 2.5 millimeters, preferably about 2.3 millimeters in diameter.

In some aspects of the invention, it is preferred that the support matrix employed can be readily dissolved to release cells without the use of trypsinization. In instances where it is desirable to remove the support matrix to liberate cells, hydrogel matrices, for example alginate and alginate-based matrices, are favored as they can be readily dissolved using sodium citrate and sodium chloride solutions.

The cell or cells can be encapsulated in a biocompatible material, so that the resulting encapsulated cells (e.g. osteogenic cells) can be administered directly to a subject patient without the need to harvest cells from the encapsulation material. For this purpose, the use of alginate or alginate-based support matrices to encapsulate cells is favored, as alginate materials are biocompatible and alginate has FDA approval. Encapsulated cells, and in particular those encapsulated in alginate or alginate based materials, can be administered directly to a patient, e.g. by injection or endoscopy.

A method or use according the invention may further comprise freezing the encapsulated cells for storage. Encapsulated cells can be frozen using standard protocols, and may be frozen in the maintenance or differentiation medium in which they were cultured. A suitable method for freezing encapsulated cells involves cryopreservation in dimethyl sulfoxide (DMSO) using a slow freezing procedure as mentioned by Stensvaag et al (Cell Transplantation 13(1): 35-44, 2004).

Methods of the invention may further comprise liberation of a cell or cells from the support matrix. The present invention therefore provides a cell or cells so obtained. Where alginate or alginate based matrices are used for encapsulation, liberation of cells can be achieved by alginate dissolution. Such gentle dissolution methods may be advantageous compared to standard enzymatic methods, such as trypsinization, which may affect the behavior of the cells in long-term cultures.

The invention also provides an encapsulated cell or cells obtainable or obtained by a cell culture method of the invention; the encapsulated cells can be multipotent, e.g. osteogenic, chondrogenic or cardiomyogenic cells, or terminally differentiated, e.g. mature osteoblasts or chondrocytes.

Further provided is the use of an encapsulated cell according to the invention as a medicament. Encapsulated osteogenic cells obtained by methods of the invention are useful in bone reconstruction, e.g. in therapeutic maxifacial surgery or in cosmetic surgery. The invention also provides the use of an encapsulated osteogenic cell as a medicament for the treatment of a disease or condition selected from osteoporosis, bone breaks, bone fractures, bone cancer, osteocarcinoma, osteogenesis imperfecta, Paget's disease, fibrous dysplasia, bone disorders associated with hearing loss, hypophosphatasia, myeloma bone disease, osteopetrosis, over-use injury to bone, sports injury to bone and periodontal (gum) disease.

Further provided is the use of an encapsulated chondrogenic cell according to the invention as a medicament for the treatment of a disease or condition selected from arthritis, a cartilage disease or disorder, cartilage repair, and cosmetic reconstructive surgery. Cartilage diseases include rheumatoid arthritis and osteoarthritis especially in articular cartilage, while disorders include congenital or hereditary defects, e.g. those requiring treatment by facial reconstruction of the nasal and septal cartilage.

Yet further provided is the use of an encapsulated osteogenic cell or cells according to the invention in the manufacture of a medicament for the treatment of a disease or condition requiring bone reconstruction, e.g. a disease or condition selected from osteoporosis, bone breaks, bone fractures, bone cancer, osteocarcinoma, osteogenesis imperfecta, Paget's disease, fibrous dysplasia, bone disorders associated with hearing loss, hypophosphatasia, myeloma bone disease, osteopetrosis; over-use injury to bone, sports injury to bone, and periodontal (gum) disease.

Additionally provided is the use of an encapsulated chondrogenic cell or cells in the manufacture of a medicament for the treatment of a disease or disorder selected from: arthritis, a cartilage disease or disorder, cartilage repair, reconstructive surgery, cosmetic reconstructive surgery, rheumatoid and osteoarthritis.

In a further aspect, the invention provides a method of treatment of a subject comprising administration of encapsulated cells according to the invention. Encapsulated osteogenic cells according to the invention can be administered to a subject to treat diseases or conditions requiring bone reconstruction, osteoporosis, bone breaks, bone fractures, bone cancer, osteocarcinoma, osteogenesis imperfecta, Paget's disease, fibrous dysplasia, bone disorders associated with hearing loss, hypophosphatasia, myeloma bone disease, osteopetrosis, over-use injury to bone, sports injury to bone, and periodontal (gum) disease. Encapsulated chondrogenic cells according to the invention can be administered to a subject to treat diseases or conditions selected from: arthritis, a cartilage disease or disorder, cartilage repair, rheumatoid and osteoarthritis.

The invention also provides a method of reconstructive surgery, which may be therapeutic or cosmetic surgery, comprising administration of an encapsulated cell or cells, preferably encapsulated osteogenic or chondrogenic cells, according to the invention.

Encapsulated cells of the invention can be formulated to provide a pharmaceutical composition comprising an encapsulated cell or cells and a pharmaceutically acceptable carrier or diluent. It is preferred that the pharmaceutical composition be formulated for administration by injection, or by endoscopy.

Also within the scope of the invention is a bone or cartilage tissue derived from an encapsulated cell of the invention, suitably provided on or in a cell scaffold. Encapsulated cells can be seeded onto, and/or impregnated into, a cell scaffold, which can then be implanted to allow the cells to grow in situ in the body. Such scaffolds are particularly useful in reconstructive surgery of bone and cartilage tissues.

The invention will now be further described by way of the following non-limiting examples which further illustrate the invention, and are not intended, nor should they be interpreted to, limit the scope of the invention.

### EXAMPLES

### Example 1

### Encapsulation of Human ESC In Alginate Beads

### Cell Culture

The process of developing the feeder layer involved primary murine embryonic fibroblast (MEF). Briefly, a female mouse (strain Swiss MF1) was sacrificed in her 13th day of pregnancy by schedule I killing. Then the embryos were pulled out and their viscera removed. Embryo carcasses were finely minced in trypsin/EDTA solution (0.05% trypsin/0.53 mM EDTA in 0.1 M PBS without calcium or magnesium; Gibco Invitrogen, Life Technologies, Paisley, UK) and seeded in culture flasks in high-glucose DMEM supplemented with 10% v/v heat-inactivated FBS, 0.1 mM MEM non-essential amino acids solution, 100 U/ml penicillin, 100 μg/ml streptomycin (all from Gibco Invitrogen, Life Technologies, Paisley, UK). When the cells reached confluence, the fibroblasts were harvested and frozen in MEF freezing medium containing 60% v/v high-glucose DMEM, 20% v/v heat-inactivated FBS (all from Gibco Invitrogen, Life Technologies, Paisley, UK) and 20% v/v dimethyl sulfoxide Hybri-Max® (DMSO) (Sigma-Aldrich, Dorset, UK). MEFs no greater than passage 3 or 4 are preferred in order to culture hESCs.

The thawed MEF cells were grown on a gelatin-coated culture surface in the same medium mentioned above, excluding penicillin and streptomycin. The MEF cells were mitotically inactivated with mitomycin C before being used as a feeder layer. The inactivated cells were then trypsinized (0.05% trypsin/0.53 mM EDTA in 0.1 M PBS without calcium or magnesium; Gibco Invitrogen, Life Technologies, Paisley, UK) and were either frozen or transfer in 6 well plate as a feeder layer for hESC growth. The MEFs were frozen in the MEF freezing medium (protocol from WiCell Research Institute Inc, Madison, July 2000).

### Culture of Human Embryonic Stem Cells

Inactivated primary MEF cells were seeded for at least one day before thawing of undifferentiated human ES cells in a medium described above. The day after, undifferentiated human H1 cells (WiCell Research Institute Inc, Madison) were thawed out and seeded on MEF cells and the protocol suggested by the supplier was used to grow the cells in an undifferentiated state. The culture medium consisted of DMEM/F12 medium supplemented with 20% v/v KNOCKOUT™ SR, 2 mM L-glutamine, 0.1 mM non-essential amino acids solution (all from Gibco Invitrogen, Life Technologies, Paisley, UK), 0.1 mM 2-mercaptoethanol (2ME) (Sigma-Aldrich, Dorset, UK) and 4 ng/ml human recombinant basic fibroblast growth factor (bFGF, FGF-2) (157 aa) (R&D Systems, Oxon, UK). The cells were fed every two days.

The growth rate of these cells was much slower than that of murine ESCs. As inactivated MEF cells died after 7-10 days in culture, HESC were transferred onto anew feeder layer every 7-10 days. After thawing of cells, it took about 4-6 weeks before obtaining a sub-confluent culture well and splitting the cells. The cells grew and maintained their undifferentiated state only when they were in a colony. Single cells did not grow Occasionally, some colonies underwent spontaneous differentiation.

Encapsulation of hESC in Alginate Beads

Undifferentiated, day 4-5, hESCs were trypsinized, and resuspended in 1.1% (w/v) low viscosity alginic acid* (Sigma, UK) and 0.1% (v/v) porcine gelatin (Sigma, UK) (all dissolved in PBS, pH 7.4) solution in room temperature. The low viscosity alginic acid is a straight-chain, hydrophilic, colloidal, polyuronic acid composed primarily of anhydro-β-D-mannuronic acid residues with 1→4 linkage. With a Pharmacia peristaltic pump [Amersham Biosciences, UK (Model P-1)], a flow rate of x20, a drop height of 30 mm [(tubing autoclaved and then sterilized with 1 M NaOH for 30 minutes and washed three times with sterile PBS)] the cell-gel solution was passed through the peristaltic pump and dropped using a 25-gauge needle (Becton Dickinson, UK) into sterile, room temperature, CaCl₂ solution [100 mM calcium chloride (CaCl₂) (Sigma, UK) and 10 mM N-(2-hydroxyethyl) piperazine-N-(2-ethane sulfonic acid) (HEPES) (Sigma, UK), in distilled water, pH 7.4]. The cell-gel solution gelled immediately on contact with the CaCl₂ solution, forming spherical beads (2.3 mm diameter after swelling). The beads remained in gently stirred CaCl₂ solution for 6-10 minutes at room temperature. The beads were washed three times in PBS and placed into maintenance medium.

Undifferentiated hESC encapsulated in alginate beads were cultured in hESC maintenance medium DMEM/F12 medium supplemented with 20% v/v KNOCKOUT™ SR, 2 mM L-glutamine, 0.1 mM non-essential amino acids solution (all from Gibco Invitrogen, Life Technologies, Paisley, UK), 0.1 mM 2-mercaptoethanol (2ME) (Sigma-Aldrich, Dorset, UK) and 4 ng/ml human recombinant basic fibroblast growth factor (bFGF, FGF-2) (157 aa) (R&D Systems, Oxon, UK). The conditions for growth were 37° C., 5% CO₂ in a humidified incubator and the beads were cultured in static conditions in standard tissue culture plastic dishes. The cells and fed every 3-4 days. Any changes on the structure and morphology were evaluated and recorded using an inverted microscope (Olympus, Southall, UK) attached with a color CoolPix 950 digital camera (Nikon, Kingston-upon-Thames, UK). The beads contained both aggregates of hESC and single hESC, single hESC cells within the beads formed colonies.

After day 130 in maintenance culture, the beads were washed twice in PBS and dissolved in order to release the cells/colonies.

A sterile depolymerization buffer was used to dissolve beads [(Ca²⁺-depletion) (50 mM tri-sodium citrate dihydrate (Fluka, UK), 77 mM sodium chloride (BDH Laboratory supplies, UK) & 10 mM HEPES)] (20) was added to PBS washed beads for 15-20 minutes while stirring gently. The solution was centrifuged at 400 g for 10 minutes and the pellet was washed with PBS and centrifuged again, at 300 g for 3 minutes.

### Histology

The 130 day old human ESC aggregates from the beads were fixed with 4% paraformaldehyde (PFA) for 1 hour at room temperature and kept in 0.1% sodium azide for short or long storage (4° C.). Prior to dehydration process, the HESC aggregates were placed in PBS for 15 minutes. They were then taken through a sequential series of increasing ethanol concentrations to remove all the water. The ethanol was then completely replaced with neat xylene to remove all traces of ethanol. The xylene was then replaced with paraffin saturated xylene at room temperature overnight. The hESC aggregates in paraffin saturated xylene were then placed in an oven (60° C.) for 20 minutes. The xylene was then completely replaced with liquid paraffin. The samples were then embedded, sectioned (4 μm) and left at room temperature overnight to adhere to Vectabonded™ (Vector Laboratories, UK) glass slides.

The cells were analyzed using immunocytochemistry. The paraffin wax was removed from the sections by immersion in xylene, decreasing ethanol concentrations and then tap water. Next, the sections were autoclaved while immersed in a tri-sodium citrate, dihydrate buffer (10 mM, pH6.0) and allowed to cool and dry in order to retrieve the antigens. The samples were then incubated with 3% (v/v) blocking goat or rabbit serum (Vector Laboratories) for 30 minutes at room temperature in 0.05% (w/v) bovine serum albumin (BSA; Sigma), 0.01% (w/v) NaN₃ (Sigma) in PBS as primary diluents.

For immunofluorescence staining, ESC marker sample kit (Chemicon, International; Cat. no. SCR002) were used according to the manufacturer protocol. The monoclonal antibodies that were used are; anti-SSEA-4, anti-TRA-1-60 and anti-TRA-1-81 (provided in the kit). For Oct-4 antibody (Santa Cruz Biotechnology), the samples were incubated with primary antibodies diluted in primary diluents (1:300) at 4° C. overnight followed by two washes and incubation with secondary antibodies (goat anti-rabbit 1:300) (Santa Cruz, International) diluted in secondary diluents consisting of 0.05% (w/v) BSAin PBS for 1 hour at room temperature in the dark. Subsequently, the samples were washed twice in PBS and mounted using Vectashield™. Preparations were viewed under IX70 fluorescence inverted microscope (Olympus, Southall, UK).

A negative control sample can be achieved by omitting the primary antibody to check for background fluorescence of the secondary antibody if used, as in indirect-2 layered fluorescent labeling. The positive sample can then be accurately interpreted with these data. The negative controls were used to position the markers on the fluorescence histograms to allow identification of the exact position of the negative populations and to estimate the amount of non-specific binding of the monoclonal or polyclonal antibodies to cell surface antigens.

For positive control, hESCs were grown on MEFs and immunostained using the ESC marker kit. The positive controls were used to identify specific binding of the monoclonal and polyclonal antibodies to cell surface antigens on positive samples.

RNA extraction and reverse transcription was also performed to analyze the cells. Total RNA was extracted from 175 days and 260 days hES cell aggregates formed in alginate beads using TRIzol reagent (Life Technologies, UK) and RNeasy Mini kit (Qiagen, UK), according to the manufacturer's instructions. Reverse-transcription-polymerase chain reaction (RT-PCR) (Invitrogen, UK) was used to synthesize cDNA from 1 μg of total RNA in a final volume of 20 μl. Oligo (dt)20 were used to prime RT reactions, which enabled the same cDNA to be PCR amplified with different sites of gene-specific primers. Negative controls were performed in the absence of cDNA template. Primers were designed using Primer Express 2 software (Applied Biosystems, UK). RT-PCR sequences are shown in Table 1.

TABLE 1
Sequences of Primers used in the Study

| | | | Annealing | Amplicon |
|---|---|---|---|---|
| Gene | Primer sequence (5′-3′) | (SEQ ID NO) | Temp. (° C.) | size (bp) |
| Oct4 | F: TCTGCAGAAAGAACTCGAGCAA | (SEQ ID NO: 1) | 54 | 127 |
| | R: AGATGGTCGTTTGGCTGAACAC | (SEQ ID NO: 2) | | |
| Nanog | F: TGCAGTTCCAGCCAAATTCTC | (SEQ ID NO: 3) | 55 | 91 |
| | R: CCTAGTGGTCTGCTGTATTACATTAAGG | (SEQ ID NO: 4) | | |
| GAPDH | F: GTTCGACAGTCAGCCGCATC | (SEQ ID NO: 5) | 54 | 182 |
| | R: GGAATTTGCCATGGGTGGA | (SEQ ID NO: 6) | | |

For housekeeping mRNA, glyceraldehyde-3-phosphate dehydrogenase (GAPDH) was used because it has been shown that, in differentiating ES cell cultures, GAPDH mRNA is more stable than other housekeeping mRNA sequences. The similarity of the primer annealing sites and amplicon sequences to other human DNA and cDNA sequences was checked by NCBI's Basic Local Alignment Search Tool (BLAST) online analysis. The paired primer annealing sites and amplicon sequence were found to be unique for the target human sequences.

In the 50 μl PCR reaction mix, the final concentration of MgCl₂ and dNTP were 3 and 10 mM, respectively. DNA amplification was performed in a Mastercycler® ep (Eppendorf AG, Germany): double-stranded DNA denaturation and the activation of AmpliTaq Gold DNA Polymerase was carried out at 94° C. for 10 min, followed by 40 cycles of template denaturation at 94° C. (5 sec), primer annealing at 55° C. (for Oct4 and GAPDH; 55° C. for Nanog) and primer extension at 72° C. (30 sec). PCR products were separated on 3% (w/v) agarose gel and visualized by ethidium bromide fluorescence and size of products approximated using 100 bp ladders (Fermentas).

Digital images of ethidium bromide-stained gels were captured using the Fluor-S Multilmager system (Bio-Rad, UK), which consists of an enclosed flat-bed UV light scanner and CCD camera, connected to a computer. Images were analyzed using Bio-Rad Quantity One software (Bio-Rad, UK), which allows detection of the individual bands and subtraction of background noise, yielding intensity values due solely to the gene-specific amplified products.

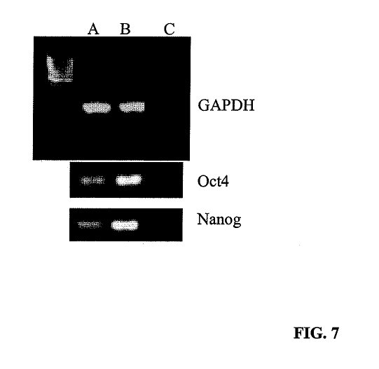

The RT-PCR analysis (FIG. 7) shows expression of pluripotent markers, Oct4 and Nanog, in both 175 day and 260 days hES cell aggregates. Lane A is 175 day old hES cell aggregates, lane B 260 day old hES cell aggregates, and lane C is a negative control. GAPDH expression was used as an internal control. These results demonstrate that pluripotency of hES cells is still maintained in hES cell aggregates for periods greater than 100 days without passage. These results also support previous immunocytochemical observations for pluripotent markers.

### Discussion and Conclusion

The results obtained demonstrate the ability of hES cells to be maintained in an undifferentiated state in the absence of feeder cells and in the absence of feeder cell conditioned medium for a period of at least 130 days. The process of hES cell encapsulation provides a physical environment that negates the requirement for such feeder cell support. The process developed enables the culture of hES cells using a method comparable to methods used for the culture of mouse ES cells. The culture procedures developed here for hES allow the hES differentiation protocols based on those currently validated using mouse ES cells, and which hitherto had not been studied in hES cells due to the lack of availability of undifferentiated ES cells in sufficient numbers for such experiments. The hES cell culture systems developed provide a valuable platform for standardized, regulatable culture systems for the development of therapeutic products using hES cells.

### Example 2

### Differentiating Single mES Cells

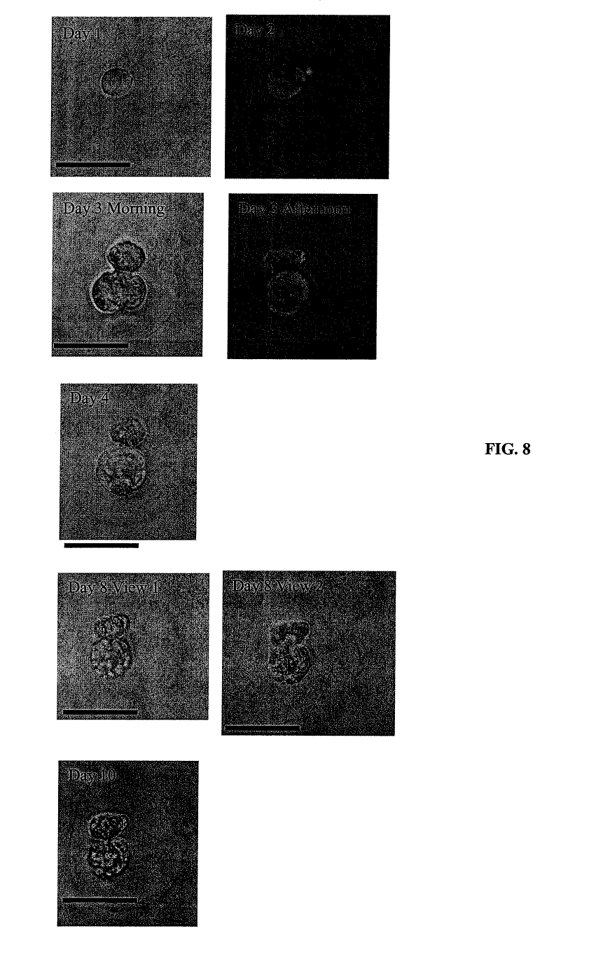

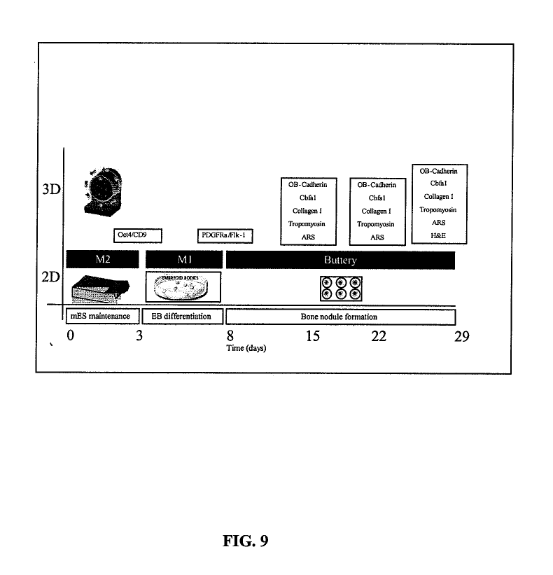

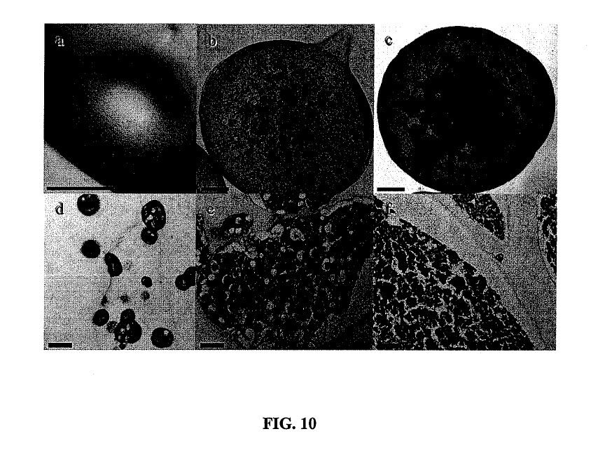

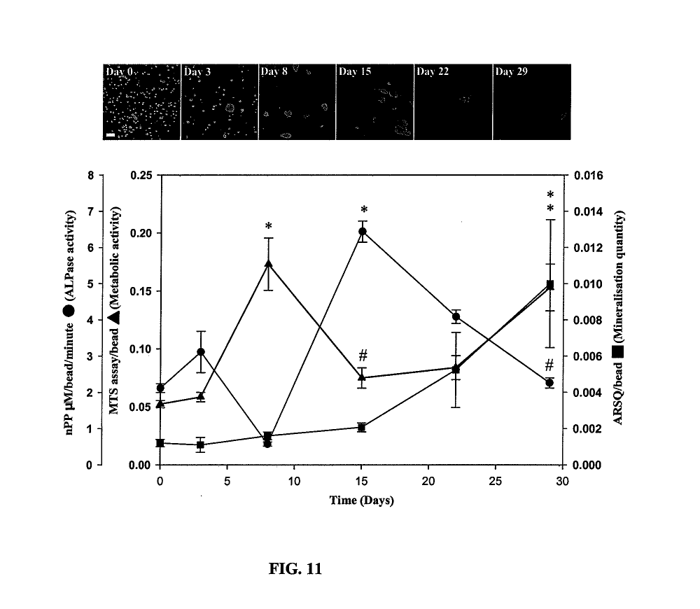

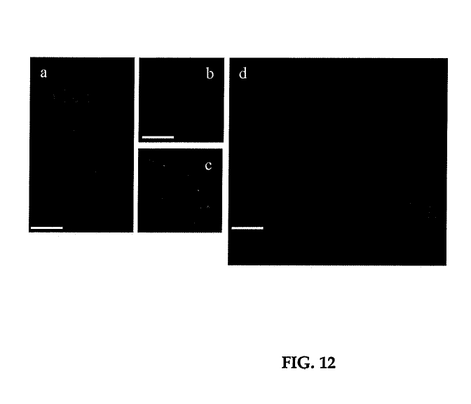

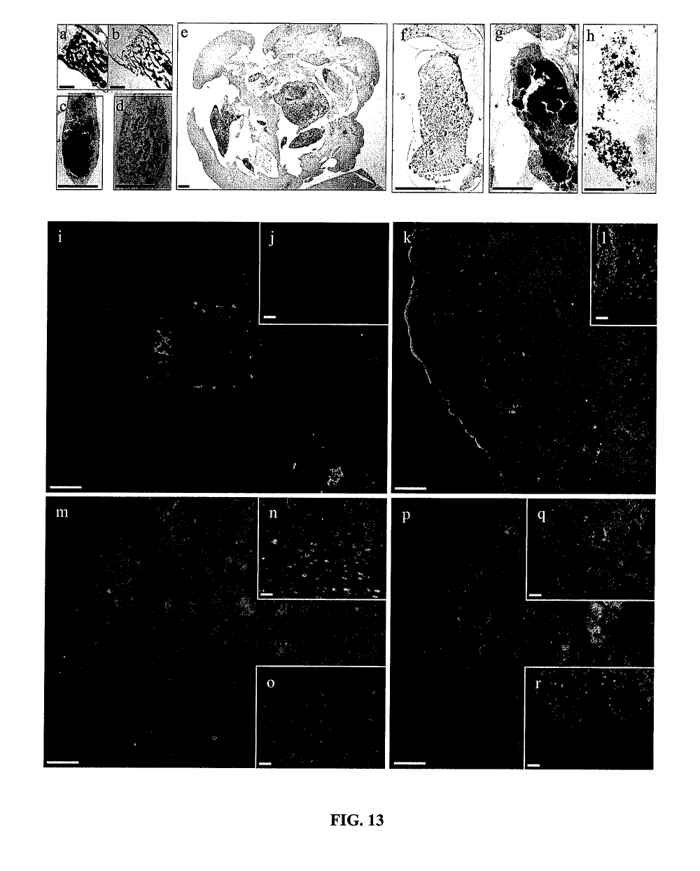

A single mES cell was encapsulated within a hydrogel bead (diameter 40-100 μm) and grown for 10 days in maintenance medium, M2 [Dulbecco's Modified Eagles Medium (DMEM), 10% (v/v) fetal calf serum, 100 units/mL penicillin and 100 μg/mL streptomycin, 2 mM L-glutamine (all supplied by Invitrogen, UK), 0.1 mM 2-Mercaptoethanol (Sigma, UK) and 1000 units/mL Esgro™ (LIF) (Chemicon, UK)]. The single ES cell undergoes division to form a small colony of cells at around 10 days (FIG. 8). These cells can be driven to differentiate into mature cells of different lineages by stimulation with established lineage-specific signals. For instance, in the case of osteogenic differentiation, the protocol described later is followed.

### Example 3

### Comparative Method, Traditional 2D mES Cell Routine Maintenance and Passage (References 2& 3)

The E14Tg2a murine embryonic stem (mES) cell line was routinely passaged on 0.1% gelatin coated tissue culture plastic in a humidified incubator set at 37° C. and 5% CO₂ (h37/5). Undifferentiated mES cells (<p20) were passaged every 2 or 3 days and fed every day with fresh M2 medium [Dulbecco's Modified Eagles Medium (DMEM), 10% (v/v) fetal calf serum, 100 units/mL penicillin and 100 μg/mL streptomycin, 2 mM L-glutamine (all supplied by Invitrogen, UK), 0.1 mM 2-Mercaptoethanol (Sigma, UK) and 1000 units/mL Esgro™ (LIF) (Chemicon, UK)]. To detach the mES, cells a desired amount of trypsin-ethylenediaminetetraacetic acid (EDTA) (TE) (Invitrogen, UK) was administered to the mES cells for 3-5 minutes (h37/5) after medium aspiration and a single wash with prewarmed PBS.

### 2D EB Formation

Embryoid body formation involved careful preparation of mES cells prior to suspension culture and is well documented (8; 9; 24; 28-30). However, empirical determination of the correct conditions before suspension was established here with the E14Tg2a cell line. Cells in monolayer culture should be ˜80% confluent by either day 2 or 3 of culture and have a very high morphological undifferentiated to differentiated ratio. The mES cells were trypsinized as normal, but clumps of 100-200 cells were visible after 2-3 minutes instead of 5 minutes trypsinization. The cells were then centrifuged at 300 g for 3 minutes at room temperature (22° C., (RT)). A confluent T75 flask, after 2 or 3 days growth in M2 medium, typically yielded around 5-7×10⁶ cells, which were resuspended in 30 mL of M1 medium [Alpha-Modified Eagles Medium (αMEM), 10% (v/v) fetal calf serum, 100 units/mL penicillin and 100 μg/mL streptomycin] and distributed evenly between two 90 mm diameter bacteriological grade petri dishes (Bibby Sterilin, UK). Clumps of 10-20 cells are essential for correct EB formation by this method, as single cell suspensions or large clumps of thousands of cells will result in erroneous 3D aggregation. On day three of EB formation (h37/5) there was a medium change, as essential growth factors had become depleted (e.g. L-glutamine) and toxic metabolites had begun to accumulate (e.g. ammonia). On day 5 of culture, the EBs were harvested by aspiration from the bacteriological plates and centrifuged at 66 g for 4 minutes. The medium was aspirated and replaced with pre-warmed PBS to wash away traces of serum. The cells were centrifuged again at 66 g for 4 minutes and the PBS was aspirated. 1 mL of TE was added to the EBs after washing for 3-5 minutes (h37/5). Prewarmed M1 media (1 mL) was then added to halt trypsinization and the cells were resuspended in the desired medium as a single cell suspension for the bone nodule forming assays.

### 2D Bone Nodule Assay

Standard bone nodule forming assays, as described previously (31), were performed using M1 medium, supplemented continuously with βadex [β-glycerophosphate at 10 mM, ascorbic acid at 50 μg/ml and dexamethasone at 1 μM (final concentrations)] from day 8 to day 29. Disaggregated EBs (dEBs) were cultured for 21 days (h37/5) with media changes every 2 or 3 days on tissue culture plastic or glass slides. The plating density of dEBs was 5.208×10³ cells per cm², with 1 μL of medium for every 25 cells.

### Example 4

### mES Alginate Bead Encapsulation

Undifferentiated murine ESCs (mESCs) were encapsulated in 1.1% (w/v) low viscosity alginic acid and 0.1% (v/v) porcine gelatin hydrogel beads (d=2.3 mm). Approximately 600 beads containing 10,000 mESCs per bead were cultured in a 50 mL horizontal aspect ratio vessel (HARV) bioreactor. The bioreactor cultures were set at a rotational speed of 17.5 rpm and cultured in maintenance medium containing leukemia inhibitory factor (LIF) for 3 days which was then replaced with EB formation medium for 5 days, followed by osteogenic medium containing L-ascorbate-2-phosphate (50 μg/mL), β-glycerophosphate (10 mM) and dexamethasone (1 μM) for a further 21 days. After 29 days in culture, an 84-fold increase in cell number per bead was observed and mineralized matrix was formed within the alginate beads. Osteogenesis was evaluated by von Kossa and Alizarin Red S staining, alkaline phosphatase activity, immunocytochemistry for osteocalcin, OB-cadherin and collagen type-I, RT-PCR and micro-computed tomography (micro-CT). These findings offer a simple and integrated bioprocess for the reproducible production of three-dimensional (3D) mineralized tissue from mESCs with potential clinical applications.

### Materials and Methods

Regarding murine ESC culture and embryoid body formation, the culture of E14Tg2a cells and formation of EBs were carried out as previously described (32). Briefly, undifferentiated mESCs (<p20) were passaged every 2-3 days and fed daily with maintenance medium consisting of Dulbecco's Modified Eagle's Medium (DMEM; Invitrogen, Paisley, UK) supplemented with 10% (v/v) foetal calf serum (FCS; Invitrogen), 100 units/mL penicillin (Invitrogen), 100 μg/mL streptomycin (Invitrogen), 2 mM L-glutamine (Invitrogen), 0.1 mM 2-mercaptoethanol (Sigma, UK), and 1000 units/mL LIF (Chemicon, Chandlers Ford, UK). EBs were disrupted and clumps (10-20 cells) were placed in EB differentiation medium consisting of alpha-Modified Eagle's Medium (αMEM; Invitrogen), 10% (v/v) FCS (Invitrogen), 100 units/mL penicillin (Invitrogen), and 100 μg/mL streptomycin (Invitrogen) in suspension for 5 days.

Mineralized tissue formation was performed, as described previously (13), using α-MEM (Invitrogen) supplemented with 50 μg/mL L-ascorbate-2-phosphate (Sigma), 10 mM β-glycerophosphate (Sigma), and 1 μM dexamethasone (Sigma) from days 8 to 29 of culture in tissue culture plastic or glass slides maintained at 37° C. and 5% CO₂. The plating density was 5.2×10³ cells/cm² and the medium was changed every 2 or 3 days.

Undifferentiated mESCs were suspended at 1.56×10⁶ cells/mL in sterile 1.1% (w/v) low viscosity alginic acid (Sigma), 0.1% (v/v) porcine gelatin (Sigma) phosphate-buffered saline solution (PBS; pH 7.4). The cell-gel solution was passed through a peristaltic pump (Model P-1; Amersham Biosciences, Amersham, UK) and dropped from 30 mm using a 25-gauge into a sterile solution of 100 mM CaCl₂, 10 mM N-(2-hydroxyethyl) piperazine-N-(2-ethane sulfonic acid) (HEPES; pH 7.4) (all from Sigma). The beads formed during gelation at room temperature for 6-10 minutes were spherical (diameter=2.3 mm after swelling). The encapsulated mESCs were cultured for 3 days in maintenance medium in 50 mL horizontal aspect ratio vessel bioreactors (Cellon, Bereldange, LUX) with daily medium changes. Each reactor contained 600 beads and was rotated at 17.5 rpm from day 0-21 of culture and at 20 rpm from day 22-29 of culture. Rotational speed was increased to compensate for the formation of mineralized tissue in the alginate beads, which resulted in the beads becoming heavier. From day 3 until day 8, the bioreactor cultures were fed with EB differentiation medium (αMEM, as previously described) which was replenished on day 6, followed by osteogenic induction on day 8 with osteogenic supplements, as described earlier (replenished every 2-3 days).

In a live/dead assay, suspended cells or alginate beads were incubated at room temperature for 30 minutes in the dark with 4 μM EthD-1 and 2 μM calcein AM solution (Invitrogen) in PBS followed by a PBS wash. Dead cells were used as a negative control.

Control 2D cell cultures grown on glass Flaskette slides (Nalgene, Hereford, UK) were fixed for 20 minutes in 4% (w/v) paraformaldehyde (PFA; BDH Laboratory Supplies) and washed in PBS. The alginate beads were fixed with 4% (v/v) paraformaldehyde (PFA; BDH Laboratory Supplies, Poole, UK) for 30 minutes at room temperature and dehydrated in increasing concentrations of ethanol followed by xylene (BDH Laboratory Supplies) prior to embedding with paraffin. The embedded samples were serially sectioned (4 μm) onto Vectabond™-coated glass slides (Vector Laboratories, Orton Southgate, UK). For immunocytochemistry, the dehydrated sections were immersed in a 10 mM tri-sodium citrate dihydrate buffer (pH 6.0; Sigma) prior to antigen retrieval by heating. Balb/c mouse bones were processed in the same manner as the alginate beads and were used as controls.

The histology of the hydrated 2D cell cultures or de-paraffinized sections of cells grown in alginate beads was examined following conventional hematoxylin/eosin staining.

Hydrated 2D cell cultures and paraffin sections were stained either with Alizarin Red S or von Kossa stain, as described elsewhere (33). Von Kossa-stained sections were counterstained with nuclear fast red, serially dehydrated, cleared in xylene and mounted in DPX. Balb/c mouse bones were used as controls and were processed in the same manner as the alginate beads.

For immunocytochemistry, hydrated 2D cell cultures or paraffin sections were immersed in a 10 mM tri-sodium citrate dihydrate buffer (pH 6.0; Sigma) and autoclaved to retrieve antigens followed by a 45 minute incubation at room temperature with 0.2% (v/v) Triton-X-100 (BDH Laboratory Supplies). As detailed in Table 2, the samples were sequentially incubated with: (a) 3% (v/v) blocking goat or rabbit serum (Vector Laboratories) for 30 minutes at room temperature in 0.05% (w/v) bovine serum albumin (BSA; Sigma), 0.01% (w/v) NaN3 (Sigma) in PBS as primary diluent; (b) primary antibody against a range of markers for stem cells and osteoblasts diluted in primary diluent at 4oC overnight; (c) secondary antibody diluted in secondary diluent [0.05% (w/v) BSA in PBS] for 1 hour at room temperature in the dark. The samples were then washed with PBS and mounted using Vectashield™ with 1.5 μg/mL 4′,6 diamidino-2-phenylindole (DAPI) (Vector Laboratories). Balb/c mouse bones were used as controls and were processed in the same manner as the alginate beads.

For reverse transcription-PCR, total RNA was extracted using the total RNA isolation kit (Qiagen Ltd, Crawley, UK). Single-stranded cDNA synthesis was performed using 1 μg of total RNA, a random primer, and AMV reverse transcriptase with an RNase inhibitor (Promega, UK). The PCR reaction buffer consisted of 1× Amplitaq Gold Buffer, 2 mM MgCl₂, 200 μM dNTPs, 1.25 units of Amplitaq Gold DNA polymerase (Applied Biosystems, Warrington, UK), and 500 nM of each primer (Invitrogen). The RT-PCR analysis was conducted, as previously described (32), using 2 μL (from 20 μL) of cDNA; the primer sequences are listed in Table 3. Positive control using MC-3T3-E1 cells cultured for 10 days in osteogenic medium. Reverse transcriptase was removed for the negative control.

TABLE 2
Antigens, Antibodies, and Blocking Serums used for Immunocytochemistry

| Antigen | Primary | Secondary | Blocking serum 1 | Blocking serum 2 |
|---|---|---|---|---|
| Oct-4 | 1:80 Rabbit | 1:80 goat anti-rabbit- | 3% Normal goat | Not applicable |
| | polyclonal (Santa | FITC (Chemicon, | serum (Vector | |
| | Cruz Biotech, Calne, | Chandlers Ford, UK) | Laboratories, UK) | |
| | UK) | | | |
| CD9 | 1:750 Rat | 1:80 goat anti-rat- | 3% Normal goat | 1.5% Normal mouse |
| | monoclonal | rhodamine. | serum (Vector | serum (Serotec, |
| | (Research | (Chemicon) | Laboratories) | Kidlington, UK) |
| | Diagnostics, | | | |
| | Concord, MA, USA) | | | |
| Flk-1 | 1:200 Mouse | 1:80 Rabbit anti-mouse | 3% Normal rabbit | 1.5% Normal mouse |
| | monoclonal (Santa | FITC (Dako, High | serum (Vector | serum (Serotec) |
| | Cruz biotech) | Wycombe, UK) | Laboratories) | |
| OB-Cadherin | 1:50 Goat polyclonal | 1:100 Rabbit-anti goat | 3% Normal rabbit | Not applicable |
| | (Santa Cruz Biotech) | FITC (Sigma) | serum (Vector | |
| | | | Laboratories) | |
| Osteocalcin | 1:50 Goat polyclonal | 1:100 Rabbit-anti goat | 3% Normal rabbit | Not applicable |
| | (Santa Cruz biotech) | FITC (Sigma) | serum (Vector | |
| | | | Laboratories) | |
| Type-I | 1:50 Rabbit | 1:100 Goat anti-rabbit- | 3% Normal goat | Not applicable |
| Collagen | Polyclonal (Santa | FITC (Chemicon) | serum (Vector | |
| | Cruz biotech) | | Laboratories) | |

TABLE 3
Sequences of Primers used in the Study

| | FWD 5′-3′ | RVS 5′-3′ | | |
|---|---|---|---|---|
| Gene | (SEQ ID NO) | (SEQ ID NO) | Length (bp) | PCR conditions |
| Gapdh | CATCACCATCTTC | ATGCCAGTGAG | 474 | 10 min 94° C., 35 cycles: |
| | CAGGAGC | CTTCCCGTC | | 94° C. 30s, 60° C. 40s, |
| | (SEQ ID NO: 7) | (SEQ ID NO: 8) | | 72° C. 60s & 10 min 72° C. |
| Cbfa-1 | CAGTTCCCAAGCA | TCAATATGGTCG | 444 | 10 min 94° C., 36 cycles: |
| | TTTCATCC | CCAAACAG | | 94° C. 60s, 45° C. 60s, |
| | (SEQ ID NO: 9) | (SEQ ID NO: 10) | | 72° C. 60s & 10 min 72° C. |
| Collagen I | GAACGGTCCACG | GGCATGTTGCTA | 167 | 10 min 94° C., 30 cycles: |
| | ATTGCATG | GGCACGAAG | | 94° C. 60s, 60° C. 60s, |
| | (SEQ ID NO: 11) | (SEQ ID NO: 12) | | 72° C. 60s & 7 min 72° C. |
| Collagen II | CTGCTCATCGCCG | AGGGGTACCAG | 432 (Splice A, | 10 min 94° C., 30 cycles: |
| | CGGTCCTA | GTTCTCCATC | early development) | 94° C. 60s, 60° C. 60s, |
| | (SEQ ID NO: 13) | (SEQ ID NO: 14) | 225 (Splice B, | 72° C. 60s & 7 min 72° C. |
| | | | mature cartilage) | |
| Osteocalcin | CGGCCCTGAGTCT | ACCTTATTGCCC | 193 | 10 min 94° C., 30 cycles: |
| (OCN) | GACAAA | TCCTGCTT | | 94° C. 60s, 60° C. 60s, |
| | (SEQ ID NO: 15) | (SEQ ID NO: 16) | | 72° C. 60s & 7 min 72° C. |

For the MTS assay, the CellTiter 96® AQueous One Solution Reagent assay (Promega, Southampton, UK) was used to assess metabolic activity throughout the culture period. Standard curves were produced using known numbers of mESCs grown in flask cultures (2D) or encapsulated in alginate beads (3D). Negative controls (no cells) were performed. All assays were done in duplicate, on three separate occasions and, for each assay, measurements were taken in quadruplicate. Briefly, mESCs cultured in 2D were incubated for 2 hours at 37° C. with 200 μL of phenol red-free maintenance medium along with 40 μL of MTS reagent in a 24 well plate. Only the 2D reaction was halted by addition of 50 μL of 10% (v/v) sodium dodecyl sulphate (SDS). Similarly, three alginate beads were selected at random, placed into separate wells of a 24 well plate, and incubated for 4 hours at 37° C. with 300 μL of phenol red-free maintenance medium and 60 μL of MTS reagent. 100 μL from each reaction were transferred into 96 well plate wells and read at 450 μm using an MRX II plate reader (Dynex Technologies, Worthing, UK).

To analyze the quantity of DNA, the total DNA content of proteinase-K-digested samples was measured using the DNA-specific dye Hoechst 33258 (Sigma) as an indirect method of evaluating cell numbers in the alginate beads. Briefly, the beads were dissolved in depolymerization buffer (20) for 20 minutes at room temperature and the cell pellet was collected after centrifugation at 400 g for 10 minutes followed by a wash with PBS. The pellets were snap frozen in liquid nitrogen and stored at −80° C. until analysis. For DNA analysis, the pellets were digested overnight at 37° C. in a 100 mM dibasic potassium phosphate (Sigma) solution containing 50 μg/mL proteinase-K (Sigma). Following heat inactivation of proteinase-K and centrifugation at 12,000 g for 10 minutes, 100 μL of supernatant was mixed with 100 μL of Hoescht 33258 solution (2 μg/mL). Finally, 100 μL aliquots were read using a MFX microtiter plate fluorometer (Dynex Technologies) with the excitation wavelength being at 365 nm and emission at 460 nm. A calibration curve was generated using highly polymerized calf-thymus DNA (Sigma). Samples were in duplicate for three independent experiments at day 0 and day 29 of culture.

Alizarin Red S (ARS) assay of mineralization of the encapsulated mESCs was quantified throughout the culture by adapting the method of Gregory et al. (34). Briefly, 100 beads were fixed with 10% (v/v) formaldehyde for 30 minutes and dissolved in depolymerization buffer (20) for 20 minutes. The cell pellet was recovered by centrifugation at 400 g for 10 minutes and was then stained in an identical fashion to the 2D cultures.

Alkaline phosphatase activity of mESCs cultured in flask cultures or encapsulated in alginate beads (n=6) was determined by incubating the cells or beads with 150 μL of alkaline-phosphatase buffer (pNPP; Sigma) and 150 μL of p-nitrophenol phosphate solution for 30 minutes at 37° C. in the dark. The reaction was stopped by adding 100 μL of 0.5N NaOH solution to each well and 100 μL from each reaction were transferred into a 96 well plate well and read at 410 nm using an MRX II plate reader (Dynex Technologies).

Images were captured using an IX70 inverted microscope (Olympus, Southall, UK) equipped with a CoolPix 950 digital camera (Nikon, Kingston-upon-Thames, UK) or a BX60 upright (Olympus) microscope equipped with an Axiocam (Zeiss). No artificial enhancement of the images was made; however, the images were cropped using Adobe Photoshop 7.0. Live/dead stained samples were imaged within 30 minutes of preparation using a Bio-Rad MRC600 confocal microscope (Bio-Rad/Zeiss, Welwyn-Garden-City, UK) and processed using the COMOS software (Bio-Rad, UK).

Micro-CT analysis was performed in order to reconstruct the 3D mineralized aggregates formed within the alginate beads using a phoenix|x-ray v|tome|x computed tomography machine (Phoenix x-ray 3D Imaging System, Fareham, UK) set at 70 kV, 160 μA and calibrated accordingly. Images were taken using one detector and rotated through 360°, each section being 6.75 μm apart. 3D reconstructions were generated using the Sixtos software, originally developed by Siemens, Germany. A negative control of alginate beads without encapsulated cells and a positive control of a Balb/c mouse pup bone chip were used.

For statistical analysis, the results were expressed as mean ±standard error of mean (SEM) and analyzed using analysis of variance (ANOVA). Statistical significance was considered at P<0.05.

### Results

Three-dimensional mineralized tissue from mESCs encapsulated in alginate hydrogels and cultured in HARV bioreactors was evaluated morphologically, phenotypically (surface and molecular) and functionally (extent of mineralization). As a control, we cultured mESCs following the traditional protocol for bone nodule formation in flask (2D) cultures replicating results shown previously (31) in order to confirm that osteogenic differentiation had occurred (data not shown).

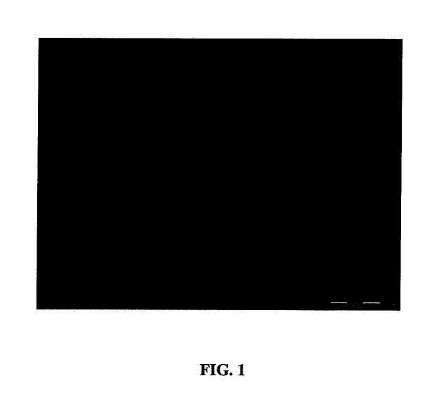

Regarding morphological characterization of encapsulated mESCs, dispersed undifferentiated mESCs were encapsulated (approximately 10,000 cells per bead) within alginate hydrogel beads of an average diameter of 2.3 mm. After 3 days of culture in maintenance medium, the mESCs that had initially been dispersed within the alginate beads formed colonies of between 4-10 cells (FIG. 1a) between 20 and 50 μm in diameter. These colonies were spherical, discoid or fusiform, and were distributed evenly around the beads but rarely located near the immediate outer bead surface (FIG. 1a). Following removal of LIF at day 3 and culture in the EB formation medium for 5 days, most colonies presented a uniform appearance and appeared to be increasing in cell number and overall size in discrete “pockets” within the alginate matrix (FIG. 1b), with the size of the colonies ranging from 50 to 400 μm in diameter. By day 22 of culture, the colonies were very tightly packed. Most of the large colonies were located towards the centre of the bead (FIG. 1c) and a zone that did not contain any cellular material was visible at the periphery. After 29 days of culture, colonies were greater than 500 μm in diameter.

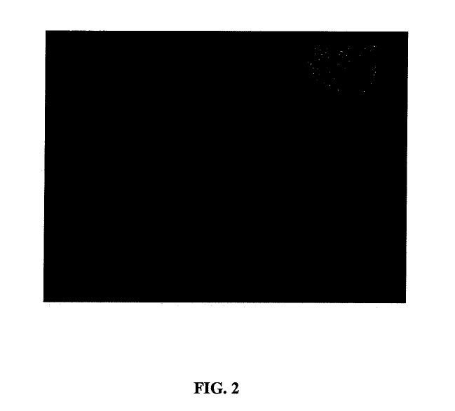

Cell viability of the encapsulated mESCs did not noticeably decrease with culture time as the colonies increased in size. At day 3, there was evidence of limited cell death, as indicated by the paucity of red cells (FIG. 2); however the majority of cells began to form discrete, live colonies. Although colony size increased with culture time, colony numbers did not increase markedly during the first 3 weeks of culture, despite the fact that viability was very high (FIG. 2). Finally, after 29 days of culture, live colonies were clearly visible in higher numbers than on day 22 and were also larger than they were earlier in culture.

The number of metabolically active, undifferentiated mESCs per bead on day 0, assessed by measuring the amount of DNA in a single bead, was found to be 10,287±228 cells per bead (mean ±SE; n=2 analyzing 150 beads for each replicate). After 29 days of culture in the HARV bioreactor there were 859,716±13,492 cells per bead (mean ±SE; n=6), representing an 84-fold increase from the start of culture. The changes in metabolic activity appeared to relate to the stage of culture, the type of medium used and the time of feeding. From day 0 to day 3, the beads were cultured in maintenance medium and the metabolic activity per bead remained unchanged (FIG. 2). On day 3 the maintenance culture medium was replaced with EB formation medium and a significant increase (p<0.05) in metabolic activity per bead was observed, as shown in FIG. 2. At day 8, the differentiation medium was introduced and the metabolic activity per bead dropped appreciably by day 15 and only increased substantially by day 29 (p<0.05) as indicated by FIG. 2. However, due to the 84-fold increase in the cell number within the alginate beads by day 29 of culture, the metabolic activity per cell does not increase.

ALPase activity and the amount of mineralization were used as indicators of osteogenic differentiation during the osteogenesis period (days 15 to 29 of culture) in osteogenic medium. ALPase activity decreased three-fold (p<0.05) between day 15 and day 29 of culture (FIG. 2). In contrast, the amount of mineralization per bead (based on absorbance at 410 μm) increased considerably (p<0.05) from 0.0021+0.0003 on day 15 to 0.0999+0.0035 (mean ±SE) on day 29, as shown in FIG. 2. The absorbance readings were normalized per bead but actual readings were taken using the mineralized contents of 100 beads per reading.

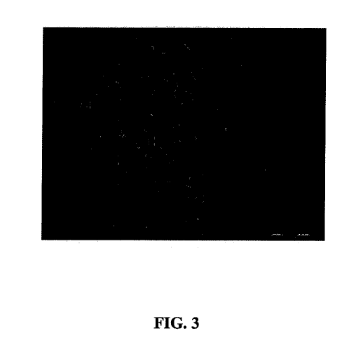

Regarding characterization of undifferentiated mESCs and EBs, retention of an undifferentiated phenotype by the encapsulated mESCs during the first 3 days of culture in maintenance medium was confirmed by expression of Oct-4 (in the nuclei) and CD9 (on the surface) at day 3 of culture (FIGS. 3a-c). Furthermore, during the EB formation stage, the encapsulated mESCs demonstrated expression of Flk-1, a marker of mesoderm, at day 8 (FIG. 3d).

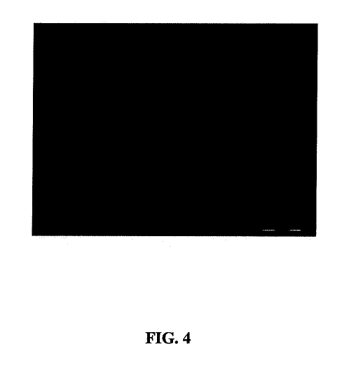

The 3D mineralized tissue formed in the alginate hydrogels from the encapsulated mESCs was extensively characterized during the osteogenesis stage of the culture (days 15-29) by examining serial sections of the alginate beads. FIGS. 4a-h demonstrates that 3D mineralized tissue was prominently formed as early as day 22 and further develops by day 29 within the alginate beads, as shown by the deep Alizarin Red S and von Kossa staining. As is evident, the samples contained a large proportion of mineralized tissue that permeated the entire section. Variations in the intensity of the staining were observed between days 22 and 29 of culture. Specifically, at the mid-phase of bone formation (day 22), the Alizarin Red S-stained tissue was uniformly red in colour (FIGS. 4c-d) but did not reach the red/black intensity found in the mouse bone positive controls (FIGS. 4a-b). Furthermore, the day 22 samples contained tissue that ranged from 100 to 300 μm in diameter, with the mineralized areas ranging from 50 to 100 μm in width. In contrast, at end of the bone formation period (day 29), the alginate beads contained larger tissue aggregations, as evidenced by the haematoxylin/eosin staining (FIGS. 4e-f); the largest tissue section having dimensions greater than 500×500 μm. Certain areas of the tissue formed appeared necrotic, however the majority were uniformly viable, as determined by viability staining (FIG. 2). Additionally, the tissue that was produced tended to occupy the centre of the beads and was highly ordered with columnar cell borders (FIG. 4e). Finally, at day 29 (FIGS. 4g-h) the mineralized tissue formed achieved the red/black Alizarin Red S staining intensity seen in positive controls (FIGS. 4a-b).

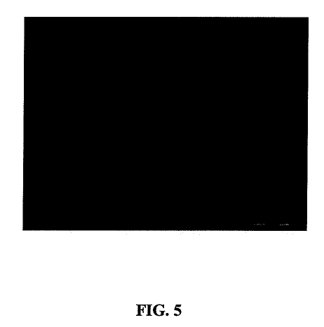

Mineralized tissue formation in the alginate hydrogels was also studied by assessing the expression of the bone-specific markers OB-cadherin, collagen type-I and osteocalcin by immunocytochemistry. Expression of OB-cadherin, which identifies osteoblasts (35), was detected on days 15, 22 and 29 of culture (FIGS. 4i-k) and was ubiquitously distributed throughout the large sections of tissue formed. Most of the staining was confined to the edges of the tissues where the cells were organized in a columnar fashion. Osteocalcin staining was detected on the periphery of the mineralized sections on the same tissue samples staining positive for OB-cadherin (FIG. 4m). Finally, collagen type-I was also detected, albeit at lower levels compared to the mouse bone positive controls, and was only visible on day 29 (FIG. 4p), which could potentially be attributed to the lower sensitivity of the polyclonal antibody used. The immunocytochemistry results were confirmed by analysis of gene expression. Specifically, RT-PCR demonstrated (FIG. 5) the expression of Cbfa-1 and collagen type-I at days 15, 22 and 29 within the beads. Collagen type-IIA, which is the transient embryonic form (21), and osteocalcin expression were found at days 15 22, and 29; on day 29 osteocalcin expression in the beads appeared to be at a similar intensity to that of positive controls (MC-3T3-E1 cells).

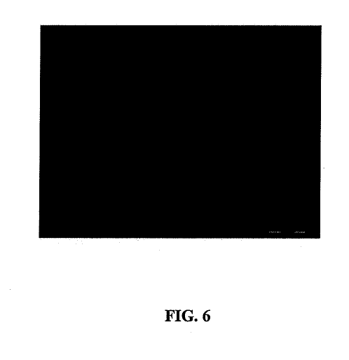

Tissue mineralization was evaluated by micro-CT analysis. Micro-CT images of negative controls consisting of alginate beads without encapsulated mESCs placed in maintenance medium produced images with very little contrast, indicating the absence of dense material able to attenuate x-rays (FIG. 6). In contrast, mineralized tissue formed within the alginate beads from the mESCs provided suitable contrast. Besides the dense bone aggregates, the superficial “crust” of the alginate beads was also detected by micro-CT outlining the periphery of the alginate beads at days 15, 22 (data not shown) and 29 (FIG. 6). The crust of the bead contained low levels of dense material (purple) and mineralized bone aggregates, within the bead itself, indicated high levels of attenuation in their centers (yellow) with decreasing attenuation as distance from the core of the bone aggregates increases. A positive control of mouse femur was imaged to compare the degree of mineralization (FIG. 6). Performing a complete scan of a randomly selected alginate bead provided a 3D reconstruction of the mineralized tissue areas within the alginate bead. On day 15, mineralized tissue aggregates were not visible, but by day 22 fourteen discrete small aggregates of less than 50 μm in diameter were visible. However on day 29, 44±7 (mean ±SE; n=2) of mineralized tissue aggregates were present ranging in size from 50 to 250 μm (FIG. 6). These mineralized aggregates were surrounded by soft tissue as seen in FIG. 4 and can be faintly recognized in FIG. 6 (red arrows) as darker regions surrounding the mineralized aggregations.

### Discussion

Embryonic stem cell culture is hindered by high maintenance since it is a fragmented process that requires trained operators and operator-dependent decisions. Currently, ESCs are cultured on tissue culture plastic as a monolayer and are subject to variations in the microenvironment due to the batch-type cultivation, frequent user intervention, and rapid exhaustion of the cultivation area. Recently, others have also highlighted the problems of traditional ESC culture and offered an integrated solution (36). In this report, we demonstrate a novel bioprocess whereby undifferentiated mESCs form 3D mineralized tissue in alginate beads in an integrated process using a HARV bioreactor without the need for interference and culture manipulation.

During the maintenance phase of mESC culture, it is imperative to sustain pluripotency and cell viability that is accomplished through the presence of LIF (4). Hence, it was vital to ensure that LIF penetrated the alginate beads, which are considered as “semi-solid” and are heterogeneous in both their calcium distribution and the arrangement of polysaccharide blocks. Calcium and alginate gradients exist in the beads, spreading from the superficial crust (highest concentration) to the bead centre (weak gelled zone) (37). These concentration gradients may explain why colonies appeared to grow 500 μm from the crust of the bead. The alginate beads prepared were permeable to proteins with a molecular weight of 68 kDa (38), which would easily allow the diffusion of LIF (39;40), for example. Each batch of 600 beads was made by gelation in the calcium chloride solution for 6 to 10 minutes. The gelation of alginate is a reaction-diffusion process in which calcium and alginate diffuse towards each other over a constant constituting boundary to form a stable structure, namely the Ca⁺⁺-alginate gel network. It seams reasonable to assume that the superficial crust on the beads always forms (as all beads remained intact) and therefore beads with a shorter exposure to the calcium chloride solution have less time to form a calcium-alginate gradient and have a larger weak gelled-zone in the center of the bead (37).

Following culture for 5 days in the EB formation medium, colony size in the alginate beads had increased dramatically, in some cases reaching 400 μm in diameter, without any significant decrease in viability. The colonies grew evenly in discrete “pockets” within the beads that have been reported to be more conducive to growth (37). Even though we encapsulated undifferentiated mESCs and did not form EBs using the traditional suspension method, expression of the Flk-1 antigen during days 3-8 in culture confirmed the development of mesoderm (23;41).

Expression of OB-cadherin early during osteogenesis (day 15) indicated the presence of osteoblasts in the 3D cultures (42). These osteoblasts were both alive (esterase activity) and metabolically active (dehydrogenase activity) at day 15. Metabolic activity fluctuated during the culture time. At the onset of osteogenic differentiation (day 8), metabolic activity per bead was high and reached a low at day 15, which correlated with ALPase activity being at its highest level whereas mineralization was near its lowest. As osteogenesis proceeded (days 15 to 29), a decrease in ALPase activity (per bead) and an increase in mineralization was observed, as has been shown in other models of osteoblast differentiation and growth (43). ALPase activity in skeletal tissues is thought to increase the local inorganic phosphate levels, destroy inhibitors of hydroxyapatite crystal growth, and aid in phosphate transport, amongst other functions (44). The latter part of osteogenesis may be the stage where osteoblasts become trapped within the secreted matrix and reduce their metabolic activity drastically in order to divert their resources to mineralization. The drop in ALPase activity, the increase in mineralization, and the low metabolic activity per cell at days 22 and 29 suggest that the cell phenotype during this period could be that of mature osteoblasts. This is further substantiated by the fact that by the end of osteogenesis (on day 29) osteocalcin, OB-cadherin and collagen type-I proteins were detected. Shimko et al (45) induced mESCs to differentiate towards bone without EB formation resulting in mineralization that, as conceded by themselves, was not considered as conventional osteogenesis. They reported that production of both osteocalcin and collagen type-I was delayed and that ALPase activity was not consistent with normal osteogenesis. In contrast, our data demonstrate conventional 3D osteogenesis occurring, as indicated by the decreasing levels of ALPase and the expression of bone-specific proteins, as early as day 15 for OB-cadherin.

Osteocalcin expression is transient in embryonic bone whereas it is one of the most abundant proteins in adult bone, binding to hydroxyapatite in a calcium-dependent manner (46;47). Woven bone is characterized by irregular bundles of collagen fibers, large and numerous osteocytes, and delayed, disorderly calcification that occurs in irregularly distributed patches (48). The presence of osteocalcin in rings and on the edges of the 3D tissue aggregates in this study, at both days 22 and 29, concurs with the micro-CT results. These observations suggest that the mineralized tissue in the alginate beads was formed by condensation of apatite crystals (bone development) and potentially at the leading edge of the osteoid front (adult lamellar bone). Our data infer that the cells, were mostly osteoblasts with proliferative capacity (49) and that hydroxyapatite had been deposited. It is accepted that differentiation from multipotent progenitors to mature osteocytes follows the proliferation, extracellular matrix development and mineralization stages with some apoptosis being seen in mature-nodules (50).

RT-PCR analysis further confirmed the presence of terminally differentiated, mineralized bone tissue, with the apparent phenotype at the endpoint of osteogenesis being mitotically active, mature osteoblasts expressing Cbfa-1, collagen type-I, and osteocalcin (49;51). Expression of embryonic collagen type-II (splice variant A) is normal during osteogenic differentiation of mESC (21;52) and, similarly, osteocalcin expression has also been previously reported from days 7 to 21 of osteogenic differentiation (53), corresponding to days 15 to 29 in this study. The lack of any mature collagen type-II (splice variant B) expression indicates that adult cartilage is not present and the bone tissue primarily consists of collagen type-I.

Adaptation of this methodology on hESCs could potentially result in their clinical implementation. Specifically, for surgical operations, such as lumbar spondylolysis, where a cancellous bone graft is required to repair a lysis of 3-4 mm (54), a single alginate bead (diameter=2.3 mm) containing 44±7 (mean ±SE, n=2) mineralized aggregates from 10,000 ESCs, could provide sufficient material to repair such a defect. In addition, it would be possible to directly inject the mineralized tissue-filled alginate hydrogels directly into the defect area (55-57). This methodology provides an attractive and beneficial alternative to traditional ESC culture and removes the bottleneck of providing large scale, 3D tissues for clinical applications. In summary, we present a simple, integrated method for the generation of 3D mineralized tissue from undifferentiated mESCs that relies on minimal operator intervention, provides reproducible results and is amenable to scale-up and online monitoring.

### Example 5

### Cryopreservation of Encapsulated Cells

Using the method described by Stensvaag et al (2004) (59), the DMSO concentration was gradually increased prior to the freezing procedure. The cryotubes were further supercooled to −7.5d° C. and nucleated. Thereafter, the samples were cooled at a rate of 0.25° C./min and stored in liquid nitrogen. The viability of the encapsulated cells was assessed using confocal microscopy quantification (CLSM) technique and a NITS assay.

The invention is further described by the following numbered paragraphs:

1. A method of cell culture comprising:
(a) providing a human embryonic stem (ES) cell encapsulated within a support matrix to form a support matrix structure, and,
(b) maintenance culture by maintaining the encapsulated cell in 3-D culture in maintenance medium.

2. A method according to paragraph 1 wherein maintenance culture is carried out in the absence of feeder cells and in the absence of feeder cell conditioned medium.

3. A method according to paragraph 1 or paragraph 2 further comprising differentiating the encapsulated cell in 3-D culture in differentiation medium in conditions suitable for cell differentiation.

4. A method according to paragraph 3 wherein the maintaining and differentiating stages are performed in the same vessel.

5. A method according to paragraph 3 or paragraph 4 wherein a stimulus for differentiation is provided.

6. A method according to paragraph 5, wherein the stimulus for differentiation comprises a stimulus for embryoid body formation.

7. A method according to paragraph 6, wherein the stimulus for embryoid body formation is removal of, or reduced, exposure to a substance that suppresses differentiation; and/or, addition of, or increased, exposure to a substance that promotes embryoid body formation.

8. A method according to any one of paragraphs 3 to 7, wherein a stimulus for differentiation to a ectodermal, endodermal or mesodermal lineage is provided.

9. A method according to any one of paragraphs 3 to 7, wherein a stimulus for differentiation to a mesodermal skeletal lineage is provided.

10. A method according to any one of paragraphs 3 to 7, wherein a stimulus for osteogenic or chondrogenic differentiation is provided.

11. A method of cell culture comprising:
(a) providing a single ES cell or a plurality of ES cells encapsulated within a support matrix to form a support matrix structure,
   (b) maintenance culture by maintaining the encapsulated cell(s) in 3-D culture in maintenance medium, in conditions suitable for ES cell maintenance,
   (c) osteogenic differentiation by differentiating the encapsulated cells in 3-D culture in differentiation medium, in conditions suitable for osteogenic differentiation.

12. A method according to paragraph 11 wherein osteogenic differentiation of the encapsulated cells comprises:
(i) incubating the encapsulated ES cells in 3-D culture in differentiation medium and providing a stimulus for embryoid body formation, then,
(ii) incubating the encapsulated cells generated in (i) in differentiation medium and providing a stimulus for osteogenic differentiation.

13. A method according to paragraph 11 wherein osteogenic differentiation of the encapsulated cells comprises:
(i) incubating the encapsulated ES cells in 3-D culture in differentiation medium, then,
(ii) incubating the encapsulated cells generated in (i) in differentiation medium and providing a stimulus for osteogenic differentiation.

14. A method according to paragraph 11 wherein osteogenic differentiation of the encapsulated cells comprises: incubating the encapsulated ES cells in differentiation medium and providing a stimulus for osteogenic differentiation.

15. A method according to any one of paragraphs 11 to 14 wherein the ES cells are of human, non-human primate, equine, canine, bovine, porcine, caprice, ovine, piscine, rodent, murine, or avian origin.

16. A method according to any preceding paragraph wherein a plurality of cells are provided encapsulated within each support matrix structure.

17. A method according to any preceding paragraph wherein a single cell is are provided encapsulated within each support matrix structure.

18. A method according to any preceding paragraph wherein in step (a) a plurality of support matrix structures are provided.

19. A method according to any preceding paragraph wherein the support matrix structure is in the form of a bead.

20. The use of a human ES cell encapsulated within a support matrix for assessing the effect of a test stimulus on cell maintenance and/or differentiation.

21. The use of a human ES cell encapsulated within a support matrix for assessing the effect of culture media and/or conditions on cell maintenance and/or differentiation.

22. A method according to paragraph 1 further comprising incubating the encapsulated cell in maintenance medium in the presence of a test compound and assessing the effect of the test compound on cell maintenance and/or differentiation.

23. A method according to paragraph 1 further comprising incubating the encapsulated cell in the presence of a test stimulus, in medium and conditions suitable for cell maintenance and/or differentiation and assessing the effect of the test stimulus on cell differentiation.

24. A method according to paragraph 1 further comprising incubating the encapsulated cell in the presence of a test medium and/or test conditions and assessing the effect of the test medium and/or test conditions, on maintenance and/or differentiation of the cell.

25. A method according to paragraph 24, wherein the cell is provided with a test stimulus and the effect of test stimulus on maintenance and/or differentiation of the cell is assessed.

26. A method according to any one of paragraphs 22 to 25, wherein in step (a) a plurality of cells is encapsulated within each support matrix structure.

27. A method according to any one of paragraphs 22 to 25, wherein in step (a) a single cell is encapsulated within each support matrix structure.

28. A method according to any one of paragraphs 22 to 27, wherein encapsulated cells are provided in an array of culture vessels.

29. A method according to paragraph 28, wherein the array of culture vessels is a multi well or multi tube array.

30. A method according to any one of paragraphs 22 to 29, wherein in step (a), a plurality of encapsulated cells is provided in each culture vessel.

31. A method according to any one of paragraphs 22 to 30, wherein in step (a), a plurality of support matrix structures are provided in each culture vessel.

32. A method according to any one of paragraphs 22 to 29, wherein in step (a), a single encapsulated cell is present in each culture vessel.

33. A method according to any one of paragraphs 22 to 32, wherein the support matrix structure is in the form of a bead.

34. A use or method according to any one of paragraphs 20 to 33, wherein the effect on cell maintenance and/or differentiation is assessed by one or more method selected from the group consisting of: microscopic examination, detection of a stage-specific antigen or antigens and detection of gene expression.

35. A method according to any preceding paragraph wherein the support matrix consists of or comprises a hydrogel.

36. A method according to any preceding paragraph wherein the support matrix consists of or comprises alginate.

37. A method according to paragraph 36 or paragraph 37 wherein the support matrix further comprises gelatin.

38. A method according to anyone of paragraphs 35 to 37, wherein the support matrix further comprises one or more material selected from the group comprising: gelatin, laminin, Bioglass™, hydroxyapatite, extracellular matrix, an extracellular matrix protein, a growth factor; an extract from another cell culture, an extract from an osteoblastic culture.

39. A method or use according to any preceding paragraph further comprising freezing the encapsulated cells.

40. A method according to any one of paragraphs 1 to 19 or paragraph 39, further comprising liberation of a cell or cells from the support matrix.

41. A cell or cells obtained by a method according to paragraph 40.

42. An encapsulated cell or cells obtainable or obtained by a method of any one of paragraphs 1 to 19.

43. An encapsulated human ES cell or cells obtained by a method of paragraph 1.

44. An encapsulated multipotent cell or cells obtained by a method of any one of paragraphs 3 to 19.

45. An encapsulated osteogenic, chondrogenic or cardiomyogenic cell or cells obtainable or obtained by a method of any one of paragraphs 3 to 19.

46. An encapsulated terminally differentiated cell or cells obtainable or obtained by a method of any one of paragraphs 3 to 19.

47. The use of an encapsulated cell according to any one of paragraphs 42 to 46, or a cell according to paragraph 41 as a medicament.

48. The use of an encapsulated osteogenic cell or cells according to paragraph 45, as a medicament for the treatment of a disease or condition requiring bone reconstruction.

49. The use of an encapsulated osteogenic cell or cells according to paragraph 45 as a medicament for the treatment of a disease or condition selected from: osteoporosis, bone breaks, bone fractures, bone cancer, osteocarcinoma, osteogenesis imperfecta, Paget's disease, fibrous dysplasia, bone disorders associated with hearing loss, hypophosphatasia, myeloma bone disease, osteopetrosis, over-use injury to bone, sports injury to bone, periodontal (gum) disease, and reconstructive surgery such as therapeutic maxifacial surgery, or cosmetic surgery.

50. The use of an encapsulated chondrogenic cell or cells according to paragraph 45 as a medicament for the treatment of a disease or condition selected from: arthritis, a cartilage disease or disorder, cartilage repair, cosmetic reconstructive surgery; rheumatoid and osteo arthritis.

51. The use of an encapsulated osteogenic cell or cells according to paragraph 45 in the manufacture of a medicament for the treatment of a disease or condition requiring bone reconstruction.

52. The use of an encapsulated osteogenic cell or cells according to paragraph 45 in the manufacture of a medicament for the treatment of a disease or condition selected from: osteoporosis; bone breaks, bone fractures, bone cancer, osteocarcinoma, osteogenesis imperfecta, Paget's disease, fibrous dysplasia, bone disorders associated with hearing loss, hypophosphatasia, myeloma bone disease, osteopetrosis, over-use injury to bone, sports injury to bone, and periodontal (gum) disease.

53. The use of encapsulated chondrogenic cell or cells according to paragraph 45 in the manufacture of a medicament for the treatment of a disease or condition selected from: arthritis, a cartilage disease or disorder, cartilage repair, reconstructive surgery; cosmetic reconstructive surgery, rheumatoid and osteo arthritis.

54. A method of treatment of a subject comprising administration of encapsulated cells according to any one of paragraphs 42 to 46 to a subject.

55. A method of treatment of a disease or condition requiring bone reconstruction comprising administration of an encapsulated osteogenic cell or cells according to paragraph 45 to a subject.

56. A method of treatment a disease or condition selected from: osteoporosis; bone breaks, bone fractures; bone cancer, osteocarcinoma, osteogenesis imperfecta, Paget's disease, fibrous dysplasia, bone disorders associated with hearing loss, hypophosphatasia, myeloma bone disease, osteopetrosis; over-use injury to bone, sports injury to bone, and periodontal (gum) disease comprising administration of an encapsulated osteogenic cell or cells according to paragraph 45.

57. A method of treatment of a disease or condition selected from: arthritis, a cartilage disease or disorder, cartilage repair, rheumatoid and osteo arthritis comprising administration of an encapsulated cell or cells according paragraph 45 to a subject.

58. A method of reconstructive surgery selected from therapeutic or cosmetic surgery comprising administration of an encapsulated cell or cells according paragraph 45 to a subject.

59. A method of reconstructive surgery selected from therapeutic or cosmetic surgery comprising administration of an encapsulated osteogenic cell or cells or chondrogenic cell or cells according paragraph 45 to a subject.

60. A pharmaceutical composition comprising an encapsulated cell or cells according to any one of paragraphs 42 to 46 and a pharmaceutically acceptable carrier or diluent.

61. A pharmaceutical composition according to paragraph 60 formulated for administration by injection, or by endoscopy.

62. A bone or cartilage tissue derived from an encapsulated cell or cells according to any one of paragraphs 42 to 46.

63. A cell scaffold having seeded on or impregnated therein encapsulated cells according to any one of paragraphs 42 to 46.

64. A. pre-filled administration device, such as a syringe, containing a pharmaceutical composition according to paragraph 62 or paragraph 63.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

### REFERENCE LIST

(1) Ulloa-Montoya F, Verfaillie C M, Hu W S. Culture systems for pluripotent stem cells. J Biosci Bioeng 2005 July; 100(1):12-27.
(2) Smith T A, Hooper M L. Medium conditioned by feeder cells inhibits the differentiation of embryonal carcinoma cultures. Exp Cell Res 1983 May; 145(2):458-62.
(3) Smith A C, Hooper M L. Buffalo rat liver cells produce a diffusible activity which inhibits the differentiation of murine embryonal carcinoma and embryonic stem cells. Dev Biol 1987 May; 121(1):1-9.
(4) Smith A G Heath J K, Donaldson D D, Wong G Q Moreau J, Stahl M, et al. Inhibition of pluripotential embryonic stem cell differentiation by purified polypeptides. Nature 1988 Dec. 15; 336(6200):688-90.
(5) Pesce M, Gross M K, Scholer H R. In line with our ancestors: Oct-4 and the mammalian germ. Bioessays 1998 Sep.; 20(9):722-32.
(6) Nichols J, Zevnik B, Anastassiadis K, Niwa H, Klewe-Nebenius D, Chambers I, et al. Formation of pluripotent stem cells in the mammalian embryo depends on the POU transcription factor Oct4. Cell 1998 Oct. 30; 95(3):379-91.
(7) Niwa H, Miyazaki J, Smith A G Quantitative expression of Oct-3/4 defines differentiation, dedifferentiation or self-renewal of ES cells. Nat Genet. 2000 Apr.; 24(4):372-6.
(8) Martin G R, Evans M J. Differentiation of clonal lines of teratocarcinoma cells: formation of embryoid bodies in vitro. Proc Natl Acad Sci USA 1975 April; 72(4): 1441-5.
(9) Desbaillets I. Embryoid Bodies: an in vitro model of mouse embryogenesis. Experimental Physiology 2000.
(10) Leahy A, Xiong J W, Kuhnert F, Stuhlmann H. Use of developmental marker genes to define temporal and spatial patterns of differentiation during embryoid body formation. J Exp Zool 1999 Jun. 15; 284(1):67-81.
(11) Sottile V, Thomson A, McWhir J. In vitro osteogenic differentiation of human ES cells. Cloning Stem Cells 2003; 5(2): 149-55.
(12) Phillips B W, Belmonte N, Vemochet C, Ailhaud Q Dani C. Compactin enhances osteogenesis in murine embryonic stem cells. Biochem Biophys Res Commun 2001 Jun. 8; 284(2):478-84.
(13) Buttery L D, Bourne S, Xynos J D, Wood H, Hughes F J, Hughes S P, et al. Differentiation of osteoblasts and in vitro bone formation from murine embryonic stem cells. Tissue Eng 2001 Feb.; 7(1):89-99.
(14) Zandstra P W, Nagy A. Stem cell bioengineering. Annu Rev Biomed Eng 2001; 3:275-305.
(15) Abbott A. Cell culture: biology's new dimension. Nature 2003 Aug. 21; 424(6951):870-2.
(16) Yamada K M, Clark K. Cell biology: survival in three dimensions. Nature 2002 Oct. 24; 419(6909):790-1.
(17) Gerecht-Nir S, Cohen S, Itskovitz-Eldor J. Bioreactor cultivation enhances the efficiency of human embryoid body (hEB) formation and differentiation. Biotechnol Bioeng 2004 Jun. 5; 86(5):493-502.
(18) Chang T M. SEMIPERMEABLE MICROCAPSULES. Science 1964 Oct. 23; 146:524-5.
(19) Lim F. Moss R D. Microencapsulation of living cells and tissues. J Pharm Sci 1981 April; 70(4):351-4.
(20) Magyar J P, Nemir M, Ehler E, Suter N, Perriard J C, Eppenberger H M. Mass production of embryoid bodies in microbeads. Ann N Y Acad Sci 2001 November; 944: 135-43.
(21) Tanaka H, Murphy C L, Murphy C, Kimura M, Kawai S, Polak J M. Chondrogenic differentiation of murine embryonic stem cells: Effects of culture conditions and dexamethasone. J Cell Biochem 2004 Oct. 15; 93(3):454.
(22) Ma H L, Hung S C, Lin S Y, Chen Y L, Lo W H. Chondrogenesis of human mesenchymal stem cells encapsulated in alginate beads. J Biomed Mater Res 2003 Feb. 1; 64A(2):273-81.
(23) Dang S M, Kyba M, Perlingeiro R, Daley G Q, Zandstra P W. Efficiency of embryoid body formation and hematopoietic development from embryonic stem cells in different culture systems. Biotechnol Bioeng 2002 May 20; 78(4):442-53.
(24) Reubinoff B E, Pera M F, Fong C Y, Trounson A, Bongso A. Embryonic stem cell lines from human blastocysts: somatic differentiation in vitro. Nat Biotechnol 2000 Apr.; 18(4):399-404.
(25) Xu C, Inokuma M S, Denham J, Golds K, Kundu P, Gold J D, et al. Feeder-free growth of undifferentiated human embryonic stem cells. Nat Biotechnol 2001 Oct.; 19(10):971-4.
(26) Beresford J N, Graves S E, Smoothy C A. Formation of mineralized nodules by bone derived cells in vitro: a model of bone formation? Am J Med Genet. 1993 Jan. 15; 45(2):163-78.
(27) Grant G T, Morris E R, Rees D A, Smith P J C, Thom D. Biological Interactions Between Polysaccharides and Divalent Cations: The Egg-Box Model. FEBS Lett 1973; 32:195-8.
(28) Hooper M, Hardy K, Handyside A, Hunter S, Monk M. HPRT-deficient (Lesch-Nyhan) mouse embryos derived from germline colonization by cultured cells. Nature 1987 Mar. 19; 326(6110):292-5.
(29) Johansson B M, Wiles M V. Evidence for involvement of activin A and bone morphogenetic protein 4 in mammalian mesoderm and hematopoietic development. Mol Cell Biol 1995 Jan.; 15(1):141-51.
(30) Wiles M V, Johansson B M. Embryonic Stem Cell Development in a Chemically Defined Medium. Exp Cell Res 1999 Feb. 25; 247(1):241-8.
(31) Bielby R C, Boccaccini A R, Polak J M, Buttery L D K. In Vitro Differentiation and In Vivo Mineralization of Osteogenic Cells Derived from Human Embryonic Stem Cells. Tissue Engineering 2004; 10(9-10):1518-25.
(32) Hwang Y S, Randle W L, Bielby R C, Polak J M, Mantalaris A. Enhanced derivation of osteogenic cells from murine embryonic stem cells following treatment with HepG2 conditioned medium and modulation of the embryoid body formation period: application to skeletal tissue engineering. Tissue Engineering 2006; X(X):X.
(33) Hale L V, Ma Y F, Santerre R F. Semi-quantitative fluorescence analysis of calcein binding as a measurement of in vitro mineralization. Calcif Tissue Int 2000 July; 67(1):80-4.
(34) Gregory C A, Gunn W Q Peister A, Prockop D J. An Alizarin red-based assay of mineralization by adherent cells in culture: comparison with cetylpyridinium chloride extraction. Anal Biochem 2004 Jun. 1; 329(1):77-84.
(35) Okazaki M, Takeshita S, Kawai S, Kikuno R, Tsujimura A, Kudo A, et al. Molecular cloning and characterization of OB-cadherin, a new member of cadherin family expressed in osteoblasts. J Biol Chem 1994 Apr. 22; 269(16):12092-8.
(36) Fok E Y, Zandstra P W Shear-Controlled Single-Step Mouse Embryonic Stem Cell Expansion and EB-Based Differentiation. Stem Cells 2005 Aug. 4.
(37) Bienaime C, Barbotin J N, Nava-Saucedo J E. How to build an adapted and bioactive cell microenvironment? A chemical interaction study of the structure of Ca-alginate matrices and their repercussion on confined cells. J Biomed Mater Res 2003 Nov. 1; 67A(2):376-88.
(38) Guo J F, Jourdian G W, MacCallum D K. Culture and growth characteristics of chondrocytes encapsulated in alginate beads. Connect Tissue Res 1989; 19(2-4):277-97.
(39) Gascan H, Godard A, Ferenz C, Naulet J, Praloran V, Peyrat M A, et al. Characterization and NH2-terminal amino acid sequence of natural human interleukin for DA cells: leukemia inhibitory factor. Differentiation inhibitory activity secreted by a T lymphoma cell line. J Biol Chem 1989 Dec. 25; 264(36):21509-15.
(40) Hilton D J, Nicola N A, Metcalf D. Specific binding of murine leukemia inhibitory factor to normal and leukemic monocytic cells. Proc Natl Acad Sci USA 1988 August; 85(16):5971-5.
(41) Kataoka H, Takakura N, Nishikawa S, Tsuchida K, Kodama H, Kunisada T, et al. Expressions of PDGF receptor alpha, c-Kit and Flk1 genes clustering in mouse chromosome 5 define distinct subsets of nascent mesodermal cells. Dev Growth Differ 1997 December; 39(6):729-40.
(42) Okazaki M, Takeshita S, Kawai S, Kikuno R, Tsujimura A, Kudo A, et al. Molecular cloning and characterization of OB-cadherin, a new member of cadherin family expressed in osteoblasts. J Biol Chem 1994 Apr. 22; 269(16):12092-8.
(43) Siggelkow H, Rebenstorff K, Kurre W. Niedhart C, Engel I, Schulz H, et al. Development of the osteoblast phenotype in primary human osteoblasts in culture: comparison with rat calvarial cells in osteoblast differentiation. J Cell Biochem 1999 Oct. 1; 75(1):22-35.
(44) Whyte M P. Hypophosphatasi. In: Bilezikian J P, Raisz L O; Rodan G A, editors. Principles of Bone Biology. 2nd ed. London: Academic Press; 2002. p. 1229-48.
(45) Shimko D A, Burks C A, Dee K C, Nauman E A. Comparison of in vitro mineralization by murine embryonic and adult stem cells cultured in an osteogenic medium. Tissue Eng 2004 Sep.; 10(9-10):1386-98.
(46) Benayahu D, Shamay A, Wientroub S. Osteocalcin (BGP), gene expression, and protein production by marrow stromal adipocytes. Biochem Biophys Res Commun 1997 Feb. 13; 231(2):442-6.
(47) Raymond M H, Schutte B C, Torner J C, Burns T L, Willing M C. Osteocalcin: genetic and physical mapping of the human gene BGLAP and its potential role in postmenopausal osteoporosis. Genomics 1999 Sep. 1; 60(2):210-7.
(48) Baron R. Anatomy and ultrastructure of bone—histogenesis, growth and remodelling. WWW 2002 [cited 2005 Jun. 28]; Available from: URL: http://www.endotext.org/parathyroid/parathyroid1/parathyroidframe1.htm
(49) Komori T, Kishimoto T. Cbfal in bone development. Curr Opin Genet Dev 1998 Aug.; 8(4):494-9.
(50) Aubin J E, Triffitt J T. Mesenchymal Stem Cells and Osteoblast Differentiation. In: Bilezikian J P, Raisz L Q Rodan G A, editors. Principles of Bone Biology (VOL I). Second ed. London: Academic Press; 2002. p. 59-81.
(51) Zur Nieden N I, Kempka Q Ahr H J. In vitro differentiation of embryonic stem cells into mineralized osteoblasts. Differentiation 2003 January; 71 (1): 18-27.
(52) Hegert C, Kramer J, Hargus Q Muller J, Guan K, Wobus A M, et al. Differentiation plasticity of chondrocytes derived from mouse embryonic stem cells. J Cell Sci 2002 Dec. 1; 115(Pt 23):4617-28.
(53) Tai G Polak J M, Bishop A E, Christodoulou I, Buttery L D. Differentiation of osteoblasts from murine embryonic stem cells by overexpression of the transcriptional factor osterix. Tissue Eng 2004 Sep.; 10(9-10):1456-66.
(54) Debnath U K, Freeman B J, Gregory P, de la H D, Kerslake R W, Webb J K. Clinical outcome and return to sport after the surgical treatment of spondylolysis in young athletes. J Bone Joint Surg Br 2003 March; 85(2):244-9.
(55) Yang W D, Cao Q, Qian Q C, Tang L H, Yang L J, Mao T Q. [A study of injectable autogenous tissue-engineered bone]. Zhonghua Kou Qiang Yi Xue Za Zhi 2003 September; 38(5):393-5.
(56) Park D J, Choi B H, Zhu S J, Huh J Y, Kim B Y, Lee S H. Injectable bone using chitosan-alginate gel/mesenchymal stem cells/BMP-2 composites. J Craniomaxillofac Surg 2005 February; 33(1):50-4.
(57) Leblond F A, Simard Q Henley N, Rocheleau B, Huet P M, Halle J P. Studies on smaller (approximately 315 microM) microcapsules: IV. Feasibility and safety of intrahepatic implantations of small alginate poly-L-lysine microcapsules. Cell Transplant 1999 May; 8(3):327-37.
(58) Lwigale P Y, Thurmond J E, Norton W N, Spooner B S, Wiens D J. Simulated microgravity and hypergravity attenuate heart tissue development in explant culture. Cells Tissues Organs 2000; 167(2-3): 171-83.
(59) Stensvaag V, Furmanek T, Lonning K, Terzis A J, Bjerkvig R, Visted T. Cryopreservation of alginate-encapsulated recombinant cells for antiangiogenic therapy. Cell Transplant 2004; 13(1):35-44.

1. A method of cell culture comprising:
(a) providing a human embryonic stem (ES) cell encapsulated within a support matrix to form a support matrix structure, and,
(b) maintaining culture by maintaining the encapsulated cell in 3-D culture in maintenance medium.

2. The method according to claim 1 wherein maintenance culture is carried out in the absence of feeder cells and in the absence of feeder cell conditioned medium.

3. The method according to claim 1 further comprising differentiating the encapsulated cell in 3-D culture in differentiation medium in conditions suitable for cell differentiation.

4. The method according to claim 3 wherein the maintaining and differentiating stages are performed in the same vessel.

5. The method according to claim 3 wherein a stimulus for differentiation is provided.

6. The method according to claim 5, wherein the stimulus for differentiation comprises a stimulus for embryoid body formation.

7. The method according to claim 6, wherein the stimulus for embryoid body formation is removal of, or reduced, exposure to a substance that suppresses differentiation; and/or, addition of, or increased, exposure to a substance that promotes embryoid body formation.

8. The method according to claim 3, wherein a stimulus for differentiation to a ectodermal, endodermal or mesodermal lineage is provided.

9. The method according to claim 3, wherein a stimulus for differentiation to a mesodermal skeletal lineage is provided.

10. The method according to claim 3, wherein a stimulus for osteogenic or chondrogenic differentiation is provided.

11. A method of cell culture comprising:
(a) providing a single ES cell or a plurality of ES cells encapsulated within a support matrix to form a support matrix structure,
(b) maintaining culture by maintaining the encapsulated cell(s) in 3-D culture in maintenance medium, in conditions suitable for ES cell maintenance,
(c) performing osteogenic differentiation by differentiating the encapsulated cells in 3-D culture in differentiation medium, in conditions suitable for osteogenic differentiation.

12. The method according to claim 11 wherein osteogenic differentiation of the encapsulated cells comprises:
(i) incubating the encapsulated ES cells in 3-D culture in differentiation medium and providing a stimulus for embryoid body formation, then,
(ii) incubating the encapsulated cells generated in (i) in differentiation medium and providing a stimulus for osteogenic differentiation.

13. The method according to claim 11 wherein osteogenic differentiation of the encapsulated cells comprises:
(i) incubating the encapsulated ES cells in 3-D culture in differentiation medium, then,
(ii) incubating the encapsulated cells generated in (i) in differentiation medium and providing a stimulus for osteogenic differentiation.

14. The method according to claim 11 wherein osteogenic differentiation of the encapsulated cells comprises: incubating the encapsulated ES cells in differentiation medium and providing a stimulus for osteogenic differentiation.

15. The method according to claim 11 wherein the ES cells are of human, non-human primate, equine, canine, bovine, porcine, caprice, ovine, piscine, rodent, murine, or avian origin.

16. The method according to claim 11 wherein the plurality of cells is provided encapsulated within each support matrix structure.

17. The method according to claim 1 wherein a single cell is are provided encapsulated within each support matrix structure.

18. The method according to claim 11 wherein in step (a) a plurality of support matrix structures are provided.

19. The method according to claim 1 wherein the support matrix structure is in the form of a bead.

20. A method of identifying a cell environment factor that is capable of modulating cell maintenance and/or cell differentiation, wherein the method comprises
(a) providing a human embryonic stem (ES) cell encapsulated within a support matrix to form a support matrix structure,
(b) maintaining culture by maintaining the encapsulated cell in 3-D culture in maintenance medium.
(c) incubating the encapsulated cell in the presence of the cell environment factor, and
(d) assessing the effect of the cell environment factor on cell maintenance and/or differentiation.

21. The method according to claim 20, wherein the cell environment factor is a test compound.

22. The method according to claim 20, wherein the cell environment factor is a test stimulus.

23. The method according to claim 20, wherein the cell environment factor is test medium and/or test conditions.

24. The method according to claim 23, wherein the cell is provided with a test stimulus and the effect of test stimulus on maintenance and/or differentiation of the cell is assessed.

25. The method according to claim 20, wherein in step (a) a plurality of cells is encapsulated within each support matrix structure.

26. The method according to claim 20, wherein in step (a) a single cell is encapsulated within each support matrix structure.

27. The method according to claim 20, wherein encapsulated cells are provided in an array of culture vessels.

28. The method according to claim 27, wherein the array of culture vessels is a multi well or multi tube array.

29. The method according to claim 27, wherein in step (a), a plurality of encapsulated cells is provided in each culture vessel.

30. The method according to claim 27, wherein in step (a), a plurality of support matrix structures are provided in each culture vessel.

31. The method according to claim 27, wherein in step (a), a single encapsulated cell is present in each culture vessel.

32. The method according to claim 20, wherein the support matrix structure is in the form of a bead.

33. The method according to claim 20, wherein the effect on cell maintenance and/or differentiation is assessed by one or more method selected from the group consisting of microscopic examination, detection of a stage-specific antigen or antigens and detection of gene expression.

34. The method according to claim 1 wherein the support matrix comprises a hydrogel.

35. The method according to claim 1 wherein the support matrix comprises alginate.

36. The method according to claim 34, wherein the support matrix further comprises one or more material selected from the group comprising gelatin, laminin, Bioglass™, hydroxyapatite, extracellular matrix, an extracellular matrix protein, a growth factor; an extract from another cell culture, an extract from an osteoblastic culture.

37. The method according to claim 35, wherein the support matrix further comprises one or more material selected from the group comprising gelatin, laminin, Bioglass™, hydroxyapatite, extracellular matrix, an extracellular matrix protein, a growth factor; an extract from another cell culture, an extract from an osteoblastic culture.

38. The method according to claim 36 wherein the support matrix further comprises gelatin.

39. The method according to claim 37 wherein the support matrix further comprises gelatin.

40. The method according to claim 1 further comprising freezing the encapsulated cells.

41. The method according to claim 1 further comprising liberation of a cell or cells from the support matrix.

42. A cell or cells obtained by the method according to claim 41.

43. An encapsulated cell or cells obtainable or obtained by the method of claim 1.

44. An encapsulated cell or cells obtainable or obtained by the method of claim 11

45. An encapsulated human ES cell or cells obtained by the method of claim 1.

46. An encapsulated multipotent cell or cells obtained by the method of claim 3.

47. An encapsulated multipotent cell or cells obtained by the method of claim 11.

48. An encapsulated osteogenic, chondrogenic or cardiomyogenic cell or cells obtainable or obtained by the method of claim 3.

49. An encapsulated osteogenic, chondrogenic or cardiomyogenic cell or cells obtainable or obtained by the method of claim 11.

50. An encapsulated terminally differentiated cell or cells obtainable or obtained by the method of claim 3.

51. An encapsulated terminally differentiated cell or cells obtainable or obtained by the method of claim 11.

52. A method of treatment of a subject comprising administration of the cells according to claim 42, to a subject.

53. A method of treatment of a subject for a disease or condition requiring bone reconstruction comprising administration of the encapsulated osteogenic cell or cells according to claim 48 to a subject.

54. A method of treatment of a subject for a disease or condition selected from osteoporosis, bone breaks, bone fractures, bone cancer, osteocarcinoma, osteogenesis imperfecta, Paget's disease, fibrous dysplasia, bone disorders associated with hearing loss, hypophosphatasia, myeloma bone disease, osteopetrosis, over-use injury to bone, sports injury to bone, and periodontal (gum) disease, wherein the method comprises administration of the encapsulated osteogenic cell or cells according to claim 48 to a subject

55. A method of treatment of a subject for a disease or condition selected from arthritis, a cartilage disease or disorder, cartilage repair, and rheumatoid and osteoarthritis, wherein the method comprises administration of the encapsulated chondrogenic cell or cells according to claim 48 to a subject

56. A method of reconstructive surgery selected from therapeutic or cosmetic surgery comprising administration of the encapsulated cell or cells according to claim 42 to a subject.

57. A method of reconstructive surgery selected from therapeutic or cosmetic surgery comprising administration of the encapsulated osteogenic cell or cells or the encapsulated chondrogenic cell or cells according claim 48 to a subject.

58. A composition comprising the encapsulated cell or cells according to claim 43 and a pharmaceutically acceptable carrier or diluent.

59. The composition according to claim 58, further comprising a pharmaceutically acceptable carrier.

60. A bone or cartilage tissue derived from the encapsulated cell or cells according to claim 43.

61. A cell scaffold having seeded on or impregnated therein encapsulated cells according to claim 43.

62. A. pre-filled administration device, such as a syringe, containing the composition according to claim 60.

63. A. pre-filled administration device, such as a syringe, containing the composition according to claim 61.


SEQUENCE LISTING (16 sequences)

SEQ ID NO: 1 - DNA, 22 bp, Artificial Sequence
  Oct4 forward primer
tctgcagaaagaactcgagcaa

SEQ ID NO: 2 - DNA, 22 bp, Artificial Sequence
  Oct4 reverse primer
agatggtcgtttggctgaacac

SEQ ID NO: 3 - DNA, 21 bp, Artificial Sequence
  Nanog forward primer
tgcagttccagccaaattctc

SEQ ID NO: 4 - DNA, 28 bp, Artificial Sequence
  Nanog reverse primer
cctagtggtctgctgtattacattaagg

SEQ ID NO: 5 - DNA, 20 bp, Artificial Sequence
  GAPDH forward primer 1
gttcgacagtcagccgcatc

SEQ ID NO: 6 - DNA, 19 bp, Artificial Sequence
  GAPDH reverse primer 1
ggaatttgccatgggtgga

SEQ ID NO: 7 - DNA, 20 bp, Artificial Sequence
  GADPH forward primer 2
catcaccatcttccaggagc

SEQ ID NO: 8 - DNA, 20 bp, Artificial sequence
  GADPH reverse primer 2
atgccagtgagcttcccgtc

SEQ ID NO: 9 - DNA, 21 bp, Artificial Sequence
  Cbfa-1 forward primer
cagttcccaagcatttcatcc

SEQ ID NO: 10 - DNA, 20 bp, Artificial Sequence
  Cbfa-1 reverse primer
tcaatatggtcgccaaacag

SEQ ID NO: 11 - DNA, 20 bp, Artificial Sequence
  Collagen I forward primer
gaacggtccacgattgcatg

SEQ ID NO: 12 - DNA, 21 bp, Artificial Sequence
  Collagen I reverse primer
ggcatgttgctaggcacgaag

SEQ ID NO: 13 - DNA, 21 bp, Artificial Sequence
  Collagen II forward primer
ctgctcatcgccgcggtccta

SEQ ID NO: 14 - DNA, 21 bp, Artificial Sequence
  Collagen II reverse primer
aggggtaccaggttctccatc

SEQ ID NO: 15 - DNA, 19 bp, Artificial Sequence
  Osteocalcin (OCN) forward primer
cggccctgagtctgacaaa

SEQ ID NO: 16 - DNA, 20 bp, Artificial Sequence
  Osteocalcin (OCN) reverse primer
accttattgccctcctgctt